(19) United States
(12) Patent Application Publication
(10) Pub. No.: US 2026/0300888 A1
(43) Pub. Date: Oct. 1, 2026

(54) SYSTEM AND METHOD FOR INCREASING EMPLOYEE PRODUCTIVITY

(71) Applicant: Mark Lamoncha, Columbiana, OH (US)

(72) Inventor: Mark Lamoncha, Columbiana, OH (US)

(21) Appl. No.: 19/694,487

(22) Filed: Jun. 1, 2026

Related U.S. Application Data
(63) Continuation-in-part of application No. 17/387,180, filed on Jul. 28, 2021.
(63) Continuation-in-part of application No. 19/438,823, filed on Jan. 2, 2026.
(63) Continuation-in-part of application No. 19/442,393, filed on Jan. 7, 2026.
(60) Provisional application No. 63/741,155, filed on Jan. 2, 2025.
(60) Provisional application No. 63/742,676, filed on Jan. 7, 2025.
(60) Provisional application No. 63/742,972, filed on Jan. 8, 2025.

(51) Int. Cl. G06Q 10/0639; G06Q 10/0833; G06Q 40/06; G06Q 50/12; G16H 10/60

(52) U.S. Cl. CPC G06Q 10/06393; G06Q 40/064; G06Q 10/0833; G06Q 50/12; G16H 10/60

(57) ABSTRACT

Systems and methods are provided for increasing employee engagement in a production activity. Performance data, including a count of output units, for the production or service activity performed by the worker is acquired on an ongoing basis while the activity is underway by way of a data acquiring device associated with the activity. A unit of output based earnings rate for the worker is retrieved from a database, and an evaluated earnings rate for the worker is generated on an ongoing basis while the activity is underway by multiplying the performance data with the unit of output based earnings rate. A graphic is displayed and updated on an ongoing basis at an electronic display associated with the worker while the activity is underway. The graphic represents the evaluated earnings rate and is automatically selected from a plurality of graphics each associated with a threshold evaluated earnings rate.

### CROSS REFERENCE TO RELATED APPLICATIONS

This application is a continuation-in-part of U.S. application Ser. No. 17/387,180 filed Jul. 28, 2021; this application is also a continuation-in-part of U.S. application Ser. No. 19/438,823 filed Jan. 2, 2026, which claims the benefit of U.S. provisional patent application Ser. No. 63/741,155 filed Jan. 2, 2025, as well as U.S. provisional patent application Ser. No. 63/742,676 filed Jan. 7, 2025; this application is also a continuation-in-part of U.S. application Ser. No. 19/442,393 filed Jan. 7, 2026, which claims the benefit of U.S. Provisional Application Ser. No. 63/742,972 filed Jan. 8, 2025; the disclosures of each of the foregoing being hereby incorporated by reference as if fully recited herein.

### TECHNICAL FIELD

Exemplary embodiments of the present invention are generally directed to a system and method for increasing employee productivity. More particularly, exemplary embodiments of the present invention are generally directed to a system and method for increasing employee productivity by illustrating the employee's pay rate directly to the employee, and preferably permits various forms of payout of earned pay.

### BACKGROUND AND SUMMARY OF THE INVENTIVE FIELD

In any workplace setting the productivity of each individual is vital to the overall success of the company. In any workplace setting, the main way to increase the profitability of the company is to measure and increase the productivity of each and every employee. There have been numerous studies and countless amounts of research performed to find ways to increase employee productivity. The results of such studies and research indicate that a one important way to increase employee productivity is to provide each and every employee with incentive to safely increase both the quantity and quality of their work output. This may be accomplished by giving each employee personal responsibility within the process for the amount and quality of work they perform.

Oftentimes, in the workplace setting, the personal responsibility of employees consists primarily of meeting certain standards, expectations or goals during their scheduled work time. Furthermore, employees typically get paid by an hourly or salary rate. Consequently, employees have no real incentive to perform at any levels higher than the minimum goal number; as they will be paid the same amount of money regardless of any excess amount of output over the minimum goal. Accordingly, these employees see a disconnect between their individual increase in output and their improvements in safety, quality and productivity.

Normally, employers provide incentives to employees by providing compensation through increases in hourly rates, salary, or periodic bonuses. However, many employees do not consider these as adequate forms of compensation. It may take weeks, months or even years of increased productivity before an employee is recognized for the fruits of his or her individual labor, if they are even so recognized. Furthermore, on a weekly or even shift basis, most employees do not have a way to evaluate if or how they may be adequately compensated if they go above the call of duty and perform above the minimum goals. Furthermore, many employees trust management to adequately compensate them for their increased productivity. This sometimes results in employees who perform just well enough not to get fired, as they are left feeling that any increased productivity will not be adequately rewarded.

Consequently, it can be understood that there is a need for a system and method for increasing employee productivity. Preferably, such a system and method would provide employees with real-time incentives to safely increase the amount and quality of their individual output. It would also be preferable that such a system and method would make each employee individually accountable for the amount of compensation they earn. Preferably, such a system and method may also provide timely monetary compensation for employees. Additionally, it is preferred that the system and method may have integrated safety and quality systems. A system and method of an exemplary embodiment of the system and method for increasing employee output may satisfy all or some of these needs/preferences.

In combination with, or separate from giving each employee personal responsibility within the process for the amount and quality of work they perform, productivity can be improved by making an activity fun and engaging. One way of making a task fun and engaging is to draw upon the individual's competitive spirit. Therefore, there is a need for a system and method which increases employee productivity through challenges.

A system and method are provided for igniting the spirit of human competition within the worker by encouraging them to safely increase productivity translating to their increased monetary compensation and their reward of meeting and beating production goals without sacrificing quality. More particularly, in one exemplary system and method employee competition is increased by illustrating the employee's pay rate in “real time” directly to a system that displays on any electronic device, stationary or portable, such as on a pay rate watch.

Generally speaking, an exemplary embodiment of the computerized system and method for increasing employee productivity provides a database for storing data, a data input device for inputting expected output parameters or other metrics into the database and a data acquiring device for acquiring actual output data or other metrics during performance of any workplace duties, preferably in real-time. The data acquiring device may be linked by an interface that transfers the acquired data to the database, where a processor may evaluate the inputted data versus the acquired data. The processor is connected to a data displaying device that may display the inputted, acquired and evaluated data, preferably in real-time.

Preferably, the system and method include a data correcting device for correcting the actual production data after the production data has been acquired. It may be preferred that an exemplary embodiment includes a network that links to a machine's PC to the machine's PLC. In some exemplary embodiments, the machine's PLC may be controlled by and interrupted by the machine's PC for safety and quality related training and/or warnings in the form of e-mails and training presentation software. In some embodiments, the machine's PC allows a user to correct actual production data and shut down the machine. It is also preferred that an embodiment may include any portable or stationary data displaying device or any pay rate watch device adapted to secure around a user's wrist and is adapted to link with the processor. In another embodiment, the system and method include a safety and quality control system that is adapted to lockout the machine whenever the employee identification number is not on an approved list of employee identification numbers stored in the database. In another embodiment, the system and method may link with a banking system for distributing a daily payroll or direct deposit of an employee's earned wages.

In other exemplary embodiments, the employee's competitive spirit is, alternatively or additionally, engaged through challenges. In exemplary systems and methods disclosed herein, the employee's increase or decrease in payrate, productivity, or other metrics may be translated into actions on an electronic display as part of a challenge which may indicate the employees progress relative to his or her peers or co-workers. The electronic display may be associated with the employee's machine. Additionally, or alternatively, the electronic display may be located in a common area and may be in electronic communication with a number of employee's machines. Each employee may compete with himself or herself. Alternatively, or additionally, each employee may compete against one another. In still other exemplary systems and methods, an animated physical device may be used instead of, or in addition to, the electronic display.

Some individuals are incentivized by future potential of their earnings even more so than the face value of the earning. By way of non-limiting example, a user earning $500 during a given shift may be more incentivized by the thought of how that $500, properly invested, could turn into more money over time. Thus, it may be desirable to provide visual feedback of earnings, and potential payout options for the same in various forms. A payout module may be placed in electronic communication with the database or other components for receiving an indication that a pay period, such as but not limited to a shift, is closing or closed. Upon receipt of such indication, the payout module may be configured to generate a user interface or display with multiple payout options for earnings of the pay period. The amount for each of the options may be determined, at least in part, based on analysis performed by a conversion module such as due to various exchange rates and may be updated, such as in substantially real time. The options displayed may be predetermined or customized. Upon selection of one of the options, the payout may be executed. The payment module may be in electronic communication with various systems for executing the desired payout including, but not limited to, an exchange system, a banking system, a brokerage system, and an ATM. In exemplary embodiments, the payouts may be recorded in one or more blocks of one or more blockchains. The blockchains may be part of a decentralized ledger, though such is not required. The new blocks of the blockchain may be authenticated by one or more user devices in exemplary embodiments.

Prior systems for monitoring employee productivity have displayed static production counts or cumulative totals but have not provided dynamic, real-time earnings rate visualizations tied directly to per-unit output data from the production machine. Furthermore, prior visual display systems have used generic progress indicators rather than automatically selecting graphics from a threshold-based set to reflect the current earnings rate tier-a feature that adds meaningful motivational feedback without supervisor intervention.

Various devices and techniques may be utilized to monitor team member performance and acquire production data, such as further provided herein.

Further features and advantages of the systems and methods disclosed herein, as well as the structure and operation of various aspects of the present disclosure, are described in detail below with reference to the accompanying figures.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is a schematic view illustrating one exemplary embodiment of a system for increasing employee productivity in a production process on a machine;

FIG. 2 is a flow diagram illustrating the steps of an exemplary embodiment of the method for increasing employee productivity in a production process on a machine;

FIG. 3 is a screenshot illustrating an exemplary embodiment of the means for displaying data;

FIG. 4 is a flow diagram illustrating exemplary logic for challenges;

FIG. 5 is a flow diagram illustrating other exemplary logic for challenges;

FIG. 6 is an exemplary challenge system for reflecting employee metrics;

FIG. 7 is another exemplary challenge system for reflecting employee metrics;

FIG. 8 is another exemplary challenge system for reflecting employee metrics;

FIG. 9A is an exemplary user interface;

FIG. 9B is another exemplary user interface;

FIG. 10 is a flow diagram illustrating exemplary logic for challenges;

FIG. 11 is a schematic view illustrating another exemplary embodiment of a system for increasing employee productivity in a production process on a machine;

FIG. 12 is an exemplary user interface for the system of FIG. 11;

FIG. 13 is a flow chart with exemplary logic for utilizing the system of FIG. 11;

FIG. 14 is a schematic view illustrating an exemplary blockchain for use with the system of FIG. 11;

FIG. 15 is a plan view of an exemplary system for indicating remaining build material for excavation and/or cleaning of parts generated by additive manufacturing;

FIG. 16 is a flow chart with exemplary logic for operating the system of FIG. 15;

FIG. 17 is a plan view of another exemplary system for indicating remaining build material for excavation and/or cleaning of parts generated by additive manufacturing;

FIG. 18 is a flow chart with exemplary logic for operating the system of FIG. 17;

FIG. 19 is a plan view of an exemplary overlay output generated by the system and/or methods of FIGS. 15-18;

FIG. 20 is a plan view of another exemplary overlay output generated by the system and/or methods of FIGS. 15-18;

FIG. 21 is a flow chart with exemplary logic for operating the system of FIG. 17;

FIG. 22 is a flow chart with exemplary logic for operating the system of FIG. 17;

FIG. 23 is a plan overview of an exemplary system for monitoring and capturing workplace performance;

FIG. 24 is flow chart with exemplary logic for the system of FIG. 23;

FIG. 25 is flow chart with other exemplary logic for the system of FIG. 23;

FIG. 26 is flow chart with other exemplary logic for the system of FIG. 23; and

FIG. 27 is flow chart with other exemplary logic for the system of FIG. 23.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

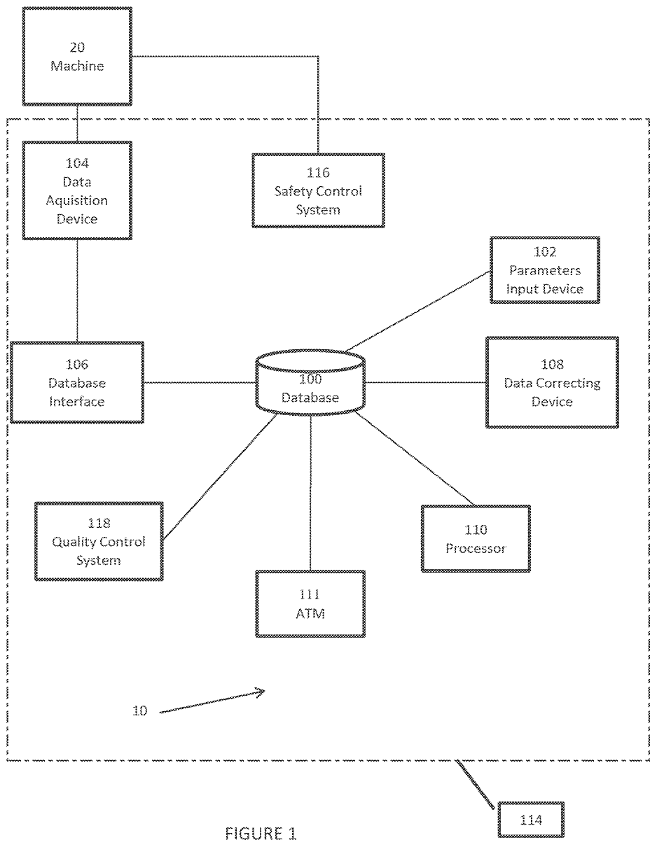

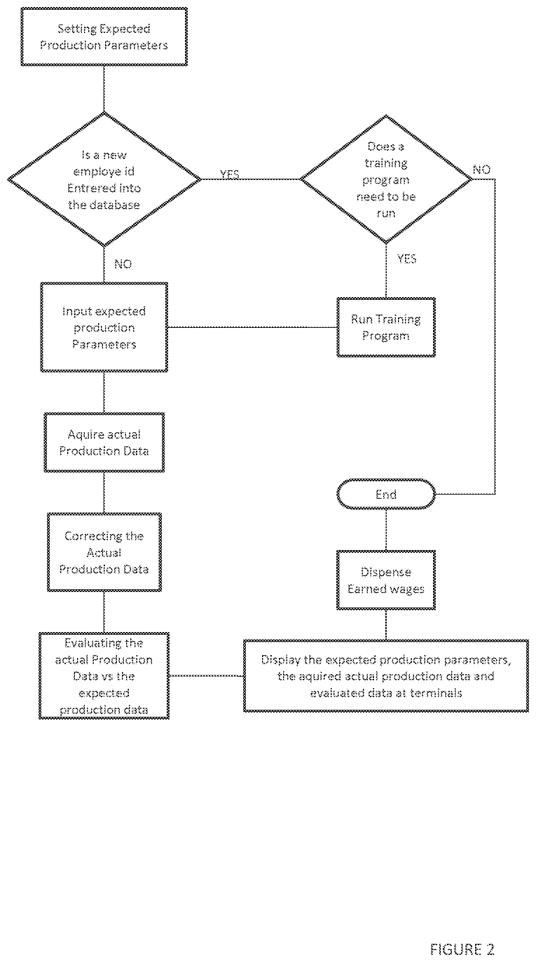

FIG. 1 depicts a schematic of one exemplary embodiment of a system 10 for increasing employee productivity in a production process on a machine 20. The machine 20 may be any type of machine for accomplishing any kind of production activity. Similarly, FIG. 2 is flow diagram illustrating the steps of an exemplary embodiment of the method for increasing employee productivity in a production process on the machine 20. As shown, this particular system 10 comprises a database 100 for storing data, a data inputting device 102 for inputting expected production parameters into the database 100, a data acquiring device 104 for acquiring actual production data from the machine 20, an interface 106 for transferring the acquired production data to the database 100, and a data correcting device for correcting actual production data 108 after the production data is acquired. Additionally, the illustrated embodiment of the system 10 includes a processor 110 for evaluating the inputted data versus the acquired data, a data displaying device 112 for displaying the inputted, acquired and evaluated data, a computer network 114 that links the machine to the database 100, and the data displaying device 112, a safety control system 116, a quality control system 118, and an automated telling machine 120, though such components are not necessarily required in all embodiments.

In the illustrated embodiment, a data inputting device 102 for inputting expected production parameters or other metrics into the database 100 for storing data is a computer terminal that is in connection with the database 100. Typically, production parameters that may be inputted in the database 100 include, but are not limited to: standard time to fabricate an individual part; standard rate of pay per part; standard pay rate per minute, hour, shift, week, month, year; standard number of parts fabricated in a minute, hour, shift, week, month, year; employee's name; employee's identification from fingerprints, retinal scan, DNA, etc.

The system 10 includes a data acquiring device 104 for acquiring actual production data or other metrics from the machine 20, which in this particular embodiment, is a programmable logic controller that is linked to multiple sensors. Typically, actual production data acquired may include, but is not limited to: total parts produced per second, minute, hour, shift, week, month, year; scrap rate per second, minute, hour, shift, week, month, year; total machine run time; runtime per part, shift, week, month, year; total machine downtime; downtime per part, shift, week, month, year; product quality data; machine safety data, etc. Typically, but not necessarily, the actual production data may be acquired automatically and in substantially real-time.

Furthermore, in this particular embodiment, an interface 106 transfers the acquired production data to the database 100. The interface 106 may be any type of connection that includes, but is not limited to: cable or telephone lines, the Internet, satellite links, etc. In this particular embodiment, the interface 106 is wired. However, in other exemplary embodiments, the interface 106 may be wireless.

Although it is preferred that an embodiment of the system 10 includes a data correcting device 108 for correcting actual production data after the production data is acquired, it is not required. In this embodiment, the data correcting device 108 is a computer terminal that is in connection with the database 100. Preferably, but not necessarily, the computer terminal is the same computer terminal used to input production parameters. In other embodiments, the data correcting device 108 may be a keypad or keyboard linked with a programmable logic controller.

This particular embodiment includes a processor 110 for evaluating the inputted production parameters versus the acquired production data to produce evaluated data. The processor 110 may execute algorithms to produce the evaluated data. The following are some examples of algorithms that may be performed by the processor 110 given the inputted production parameters and the acquired data. These examples are in no way limiting the capability of the numerous different algorithms the processor 110 may execute. One simple example of a typical algorithm executed by the processor may produce the total realized pay for an employee for a shift that could be set forth in a relationship such as, but not limited to:

$$TRPS = X * TPPS$$

where:
TRPS is the total realized pay for an employee for a shift;
X is the standard pay rate per part; and
TPPS is the total parts produced for a shift.

In this example, the standard pay rate per part would be inputted into the database 100 as one of the performance parameters and the total parts produced for a shift would be actual data acquired from the machine during the production process.

In another example, the algorithm may produce the difference between expected pay per shift and the realized pay per shift that could be set forth in a relationship such as, but not limited to:

$$DEPRP = X * TPPS - EPS$$

where:
DEPRP is the difference between the expected pay and realized pay per shift
X is the standard pay rate per part;
TPPS is the total parts produced for a shift; and
EPS is the expected pay per shift.

In this example, the standard pay rate per part and the expected pay per shift would be inputted into the database 100 as one of the performance parameters and the total parts produced for a shift would be actual data acquired from the machine during the production process.

In another example, the algorithm may produce the realized hourly rate by an employee that could be set forth in a relationship such as, but not limited to:

$$RHR = [ TPPS / T ] * X$$

where:
RHR is the realized hourly rate
TPPS is the total parts produced for a shift;
T is total time in hours; and
X is the standard pay rate per part.
   In this example, the standard pay rate per part would be inputted into the database 100 as one of the performance parameters and the total parts produced for a shift and the total time in hours would be actual data acquired from the machine during the production process.

Normally, the processor 110 may produce evaluated data including, but is not limited to: difference between expected pay and realized pay per second, minute, hour, shift, week, month, year; realized pay per minute, hour, shift, week, month year; realized pay-rate per hour, shift, week, month, year; difference between expected and realized parts produced per minute, hour, shift, week, month, year; quality data, and safety data. Typically, but necessarily, the evaluated data is produced automatically and in real-time.

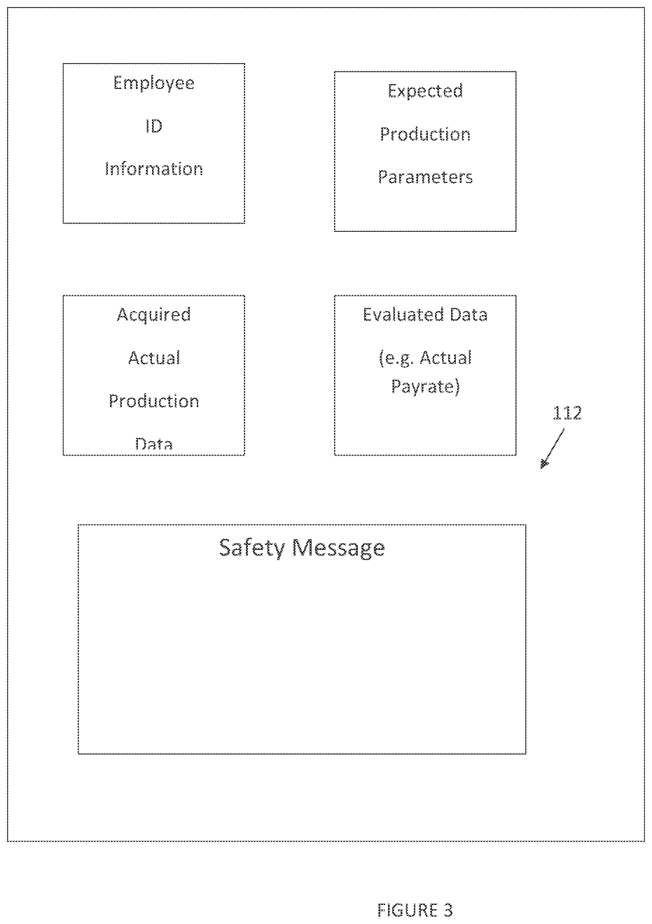

In one particular embodiment, the system 10 includes a data displaying device 112 that may display the inputted, acquired and evaluated data that is adapted to connect to the database 100. An example of a screenshot of an exemplary embodiment of the data displaying device is illustrated in FIG. 3. In this particular embodiment, the means for displaying 112 is an electronic display. Preferably, but not necessarily, the data displaying device 112 is located within visible sight of the employee operating the machine 20 to help provide incentive to the employee to increase both production rate and quality. In this regard, the data displaying device 112 may be a portable device that is adapted to connect to the database 100 using a wireless network. Preferably, but not necessarily, the portable display device may be adapted to secure around an individual's wrist, the result being a device that may resemble a wristwatch. In another embodiment, the portable device may be adapted to secure to an individual's belt. However, in other embodiments, the data displaying device 112 may be any type of display screen that may be adapted to connect to the database 100 through a wireless or wired connection. In one exemplary embodiment, the data displaying device 112 may be a monitor for a computer. In some exemplary embodiments, the data displaying device 112 is engaged with and adjacent to the machine 20 on an assembly line or in the employee's workstation to ignite the spirit of human competition within the worker by encouraging them to safely increase productivity translating to their increased monetary compensation and their reward of meeting and beating production goals without sacrificing quality.

In whatever form, all the components of the system 10 are preferably linked by a network 114. However, the network 114 may link any and/or all components of the system 10. One skilled in the art would realize that there are many different ways to network the components, including, but not limited to: cable or telephone lines, the Internet, satellite links. In this particular embodiment, the network is wireless. However, in other exemplary embodiments, the network 114 may be wired and/or wireless, depending upon the design factors and other considerations, as would be understood by one skilled in the art.

In this particular embodiment, the system 10 includes a safety control system 116 adapted to communicate with the machine. Although the safety control system 116 is preferred, other exemplary embodiments of the system 10 may not include such a safety control system 116. In one particular embodiment, the safety control system 116 may have a terminal wherein an individual may input an individual identification code. In this embodiment, depending upon the identification code entered, the machine may lockout whenever the employee identification number is not on a secured list in the database.

In other exemplary embodiments, the safety and quality control system 116 may control a machine's PLC and interrupt operation of the machine. In this particular embodiment, the safety control system 116 is controlled by a PC. In some embodiments, the safety control system 116 may interrupt the machine's PC for safety or quality related training and/or warnings in the form of emails and training presentation software. In one example, at the start of a shift, the safety control system 116 may walk a worker through a training exercise before the worker may start working. In another example, an administrator or another authorized individual may send the machine's PC an email message that must be read by a worker before the machine will start or continue operating.

In order to provide timely incentive rewards for employees, a banking system 111 for distributing a daily payroll or direct deposit of an employee's earned wages may be linked with the system 10 to provide an amount of money earned by the employee. In this particular embodiment, an individual may obtain the amount of money they earned from an automated telling machine at the end of each production shift. However, in other embodiments, the banking system 111 may allow the employee to obtain the amount of money they earned at varying time intervals, such as, but not limited to: each hour, daily, weekly, monthly, yearly or each break period. In yet another embodiment, the banking system 111 may provide other forms of compensation by itself, or in addition to monetary compensation. One example may be where the banking system 111 may provide employees with tokens that the employee may spend at a company store. In other embodiments, the banking system 111 may directly deposit the employee's earned wages into a predetermined banking or checking account.

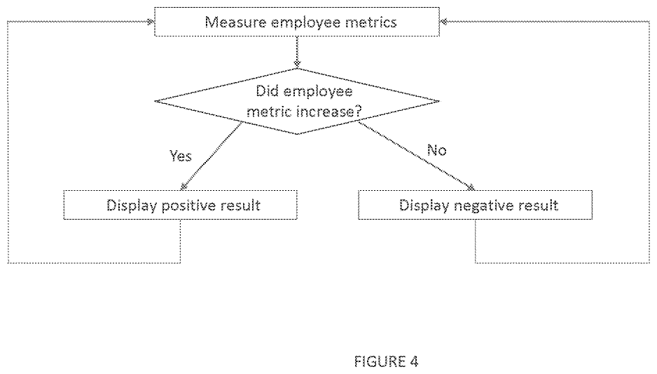

FIG. 4 is a flow diagram illustrating exemplary logic for the challenge system 10. Various employee metrics may be measured. For example, without limitation, such metrics may be measured by way of the data acquisition device 104 and may comprise actual production data. Such actual production data may include, but is not limited do: total parts produced per second, minute, hour, shift, week, month, year; scrap rate per second, minute, hour shift, week, month, year; total machine run time; runtime per part, shift, week, month, year; total machine downtime; downtime per part, shift, week, month, year; product quality data; machine safety data, etc. Additionally, or alternatively, such metrics may be measured by way of the processor 110 and may comprise real-time-pay rate. Such metrics may be measured periodically, continuously, at regular or irregular intervals, and/or in substantially real time. The measured employee metrics may be stored at the database 100, though such is not required. In still other exemplary embodiments, the metric may be compared against one or more-predetermined thresholds.

The measured employee metrics may be compared against previously measured metrics. Such comparison may be made at the processor 110, though such is not required. If the measured metric has increased as compared to a previously measured metric, a positive result may be displayed. If the measured metric has decreased, a negative result may be displayed. However, it is notable that it may be desirable to decrease some metrics and undesirable to increase some metrics. In such cases, a positive result may be displayed where a measured metric has decreased and a negative result may be displayed where a measured metric has increased. An example of such a situation is the production of defective units.

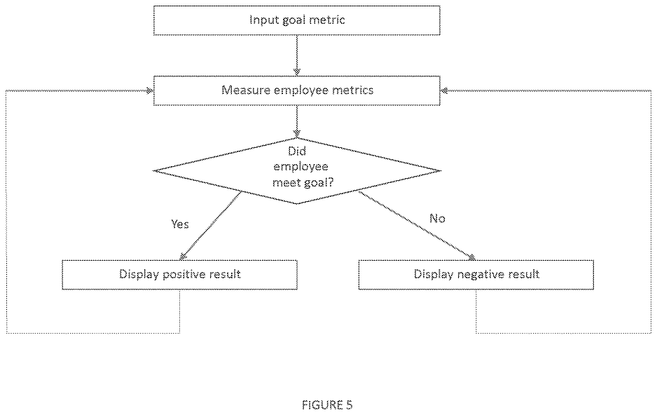

FIG. 5 illustrates other exemplary logic for the challenge system 10. A goal metric may be inputted. In exemplary embodiments, the goal metric may be inputted by way of the data input device 102 and may be stored at the database 100, though such is not required. The measured employee metrics may be compared against the goal metric. Such comparison may be made at the processor 110, though such is not required. If the measured metric has met or exceeded the goal, a positive result may be displayed. If the measured metric has not met the goal, a negative result may be displayed. However, it is notable that the goal may be a minimum, maximum, rate over time, relative goal, some combination thereof, or the like. It is further notable that the goal may be one of the employee's previously measured metrics. In still other exemplary embodiments, the metric may be compared against one or more-predetermined thresholds.

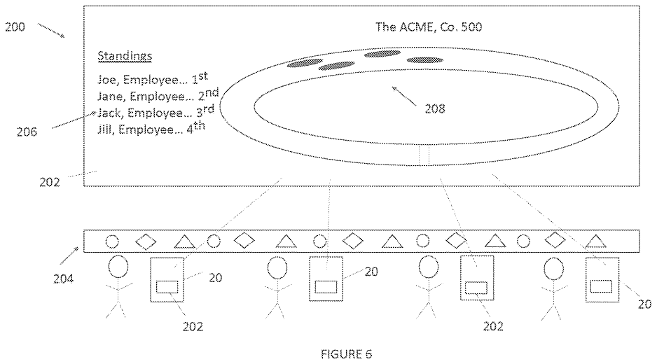

FIG. 6 is an exemplary challenge system 200 for reflecting employee metrics and/or progress relative to peers or co-workers. The challenge system 200 may comprise one or more electronic displays 202. In exemplary embodiments, an electronic display 202 may be provided on each machine 20. Alternatively, or additionally, a single electronic display 202 may be placed in electronic communication with a number of machines 20. In the illustrated example, which is merely exemplary and is not intended to be limiting, each machine 20 is associated with a given assembly line 204 and is placed in electronic communication with the challenge system 200 comprising the electronic display 202 such that all employees working on the assembly line 204 may compete against one another. The challenge system 202 may be configured to receive the metrics and determine whether a positive or negative result should be displayed. Alternatively, or additionally, the challenge system 202 may instead receive a notification of whether a positive or negative result should be displayed. For example, without limitation, such determination of a positive or negative result may be made at the processor 110.

The challenge system 200 may be configured to display the positive or negative result for each employee. In exemplary embodiments, such updates may be made in substantially real-time so that the employees receive immediate feedback regarding their work. Such positive and negative results may be reflected in a point system, a ranking, a movement or action, some combination thereof, or the like.

For example, without limitation, the electronic display 202 may be configured to display a simulated race. As positive and negative results are determined, the employee's relative standings 206 may be changed and the same may be reflected on a simulated race track 208. In this way, employees may compete against one another. The electronic display 202 may be placed in a common area such as on the ceiling, on a wall, in a break room, on the factory floor, some combination thereof, or the like. Of course, this is just one example which is not intended to be limiting. For example, without limitation, the simulated race may reflect each employee's real-time-pay rate, though any metric is contemplated.

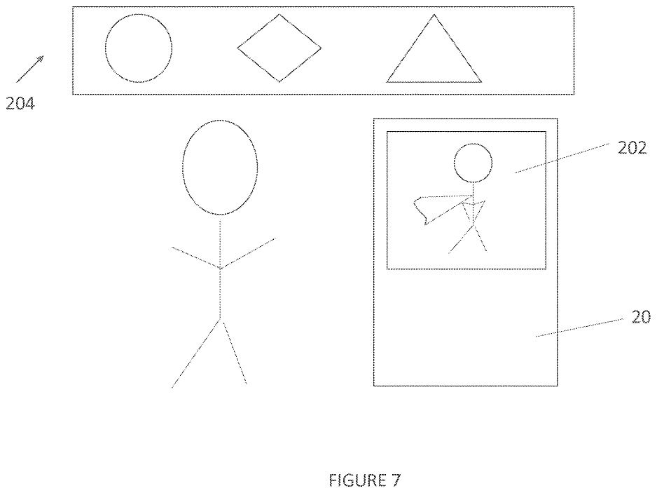

FIG. 7 is another exemplary challenge system 200 for reflecting employee metrics. In the illustrated embodiment, the electronic display 202 is provided on, or associated with, an individual machine 20. As positive or negative results are determined, the challenge system 200 may be configured to display positive and negative actions on the electronic display 202. In exemplary embodiments, an action hero may complete various tasks, gain components of a hero's outfit, or the like. Of course, this is just one example and is not intended to be limiting. In this way, the employee may compete against himself or herself or some predetermined criteria. For example, without limitation, the action hero's outfit and/or actions may reflect the employee's real-time-pay rate, though any metric is contemplated.

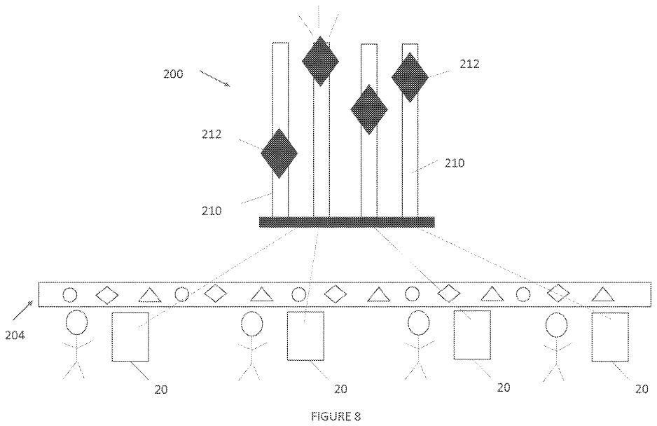

FIG. 8 is another exemplary challenge system 200 for reflecting employee metrics. The challenge system 200 may comprise an animated physical system configured to reflect the positive or negative results. In the illustrated embodiment, a number of poles 210 are provided, each having an object 212. As positive or negative results are determined, the challenge system 200 may be configured to move the object 212 up or down the pole to reflect the positive and negative results. In exemplary embodiments, a pole 210 comprising an object 212 may be provided for each machine 20 in communication with the challenge system 200 such that the employees may compete against one another. In other exemplary embodiments, a single pole 210 and object 212 may be provided for a group of machines 20 and/or employees such that employee's collective efforts may be reflected against themselves or against another group of machines 20 and/or employees. Of course, the use of poles 210 and objects 212 are merely exemplary and is not intended to be limiting. For example, in other exemplary embodiments, an animatronic person, event, object, animal, some combination thereof, or the like, may be utilized. For example, without limitation, the movement of the object 212 may reflect the employee's real-time-pay rate, though any metric is contemplated.

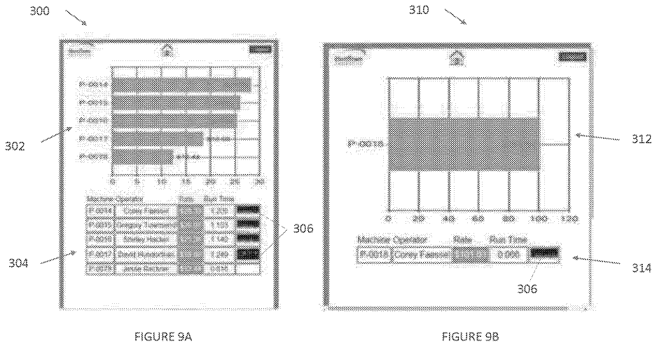

FIG. 9A is an exemplary user interface 300 for use with the challenge system 200. The illustrated user interface 300 may be utilized, for example without limitation, on the display 202. The user interface 300 may comprise a ranking of employees 304. The ranking 304 may list the machine 20 associated with the employee, the employee's name, the employee's real-time-pay rate, the amount of time the employee has been working, and a graphic. The ranking 304 may be organized to rank machines 20 and/or employees from highest to lowest in terms of their real-time-pay rate. The information in the ranking 304 may be updated periodically, or in substantially real-time. The graphic may be updated to reflect the real-time-pay rate. For example, without limitation, the graphic may comprise flames when the real-time-pay rate reaches a first pre-determined threshold, the graphic may comprise fireworks when the real-time-pay rate reaches a second pre-determined threshold, and the graphic may comprise a photo of the employee when the real-time-pay rate reaches a third predetermined threshold.

The user interface 300 may further comprise a visualization 302 illustrating the ranking 304. For example, the visualization 302 may comprise a chart, such as but not limited to, a bar chart indicating the machine 20 and/or the employee's name and the associated real-time-pay rate. The visualization 302 may be organized to rank machines 20 and/or employees from highest to lowest in terms of their real-time-pay rate. Any type of kind of visualization 302 is contemplated.

FIG. 9B is another exemplary user interface 310. In exemplary embodiments, without limitation, the user interface 310 may be displayed on an individual machine 20. The user interface 310 may, but is not required to, be similar to the user interface 300 but with information 314 pertaining to a single machine 20 or employee. For example, without limitation, the user interface 310 may comprise the machine 20 number, the employee name, the employee's real-time-pay rate, the machine's 20 run time, and a graphic 306 reflecting the real-time-pay rate. The user interface 310 may further comprise a visualization 312 illustrating the employee's real-time-pay rate. The visualization 312 may be, but is not limited to, a bar chart. Any type of kind of visualization 312 is contemplated. In exemplary embodiments, the user interface 310 may be displayed on an individual machine 20 while the user interface 300 is displayed on the display 202, though such is not required. In exemplary embodiments, both the user interfaces 300 and 310 are controlled by the challenge system 200, though such is not required.

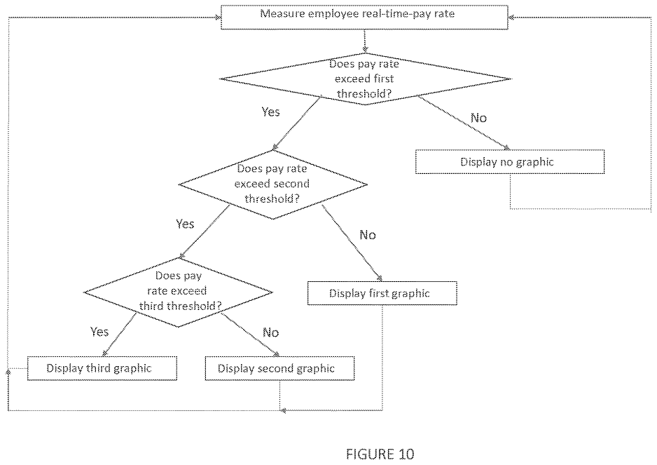

FIG. 10 is a flow diagram illustrating exemplary logic for challenges. Such challenges may, for example, indicate the employee's progress or metrics relative to peers or co-workers. The real-time-pay rate for the employee and/or machine 20 may be determined. If the real-time-pay rate exceeds a first threshold, a first graphic 306 may be displayed. If the real-time-pay rate exceeds a second threshold, a second graphic 306 may be displayed. If the real-time-pay rate exceeds a third threshold, a third graphic 306 may be displayed. Any number of pre-determined thresholds are contemplated and any number of graphics 306 of any kind to be displayed are contemplated. In other exemplary embodiments, other actions may be triggered at such thresholds including alerts sent to management, bonuses, achievement awards, and the like.

In exemplary embodiments, achievement in the challenge system 200 may be translated into incentives for the employee such as, but not limited to, increase in pay, bonuses, extra break time, dress down days, vacation days, or the like. It is notable that one, or a number of metrics, may be measured and compared. A positive or negative result may be provided based on each individual metric, or all metrics. Certain metrics may be weighted more of less compared to other metrics. Furthermore, different aspects of the challenge system 200 may be changed based on whether positive or negative results are found for particular metrics. For example, without limitation, an employee's virtual car in a race may be made to go faster as productivity increases, but may have a simulated tire blowout if a defective part is assembled that fails inspection. Of course, this is just one example. Such incentives may be provided by way of the banking system 111, though such is not required.

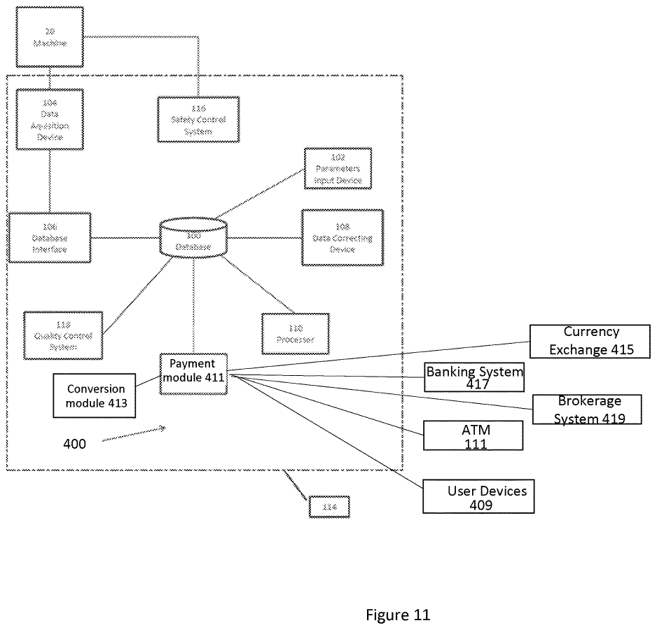

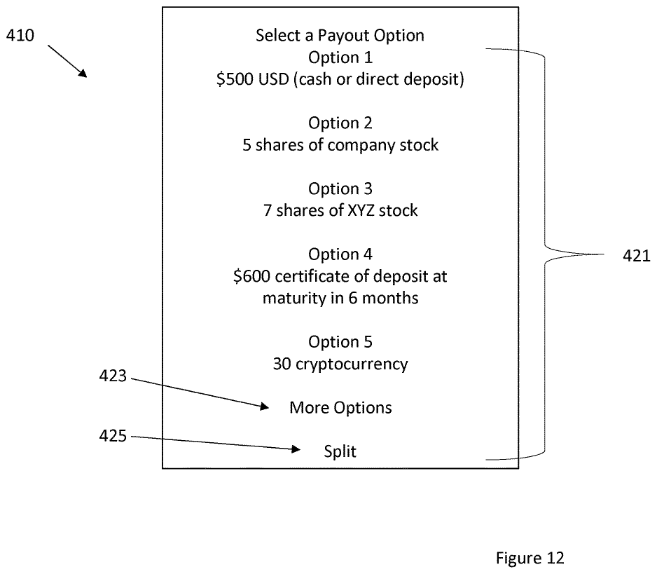

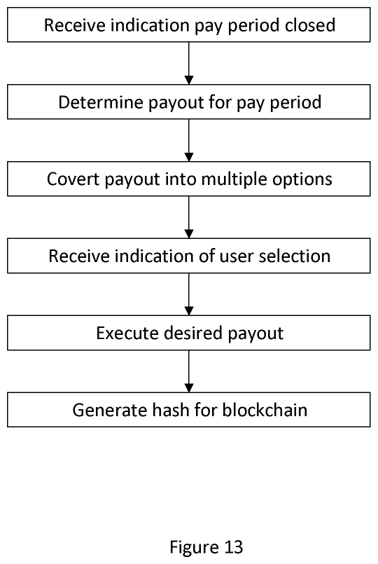

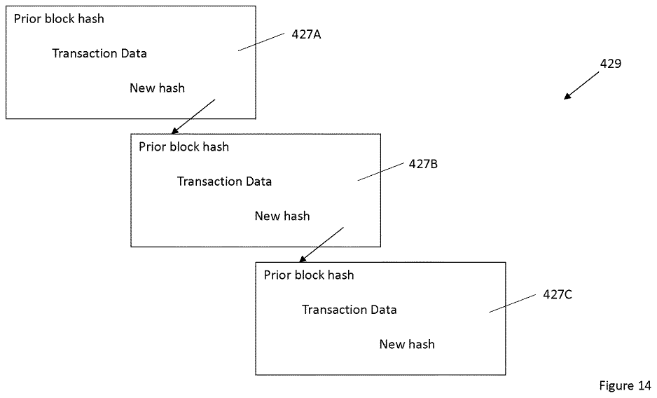

FIG. 11 through FIG. 14 illustrate another exemplary system 400 which permits a user to select one or more alternate forms of payout of earned compensation. A payout module 411 may be in electronic communication with the database 100 or other components of the system 400, such as but not limited to the processor 110. Such electronic communication may be direct or indirect. Upon completion of a pay period, such as a shift, day, week, bi-weekly period, etc., the payout module 411 may be configured to determine, or may receive indication that, the pay period has ended or is ending. Upon receipt of such indication, the payout module 411 may be configured to determine a total payout earned for each associated employee or other worker for the pay period, such as by systems and techniques shown and/or described herein. The payout module 411 may be configured to generate a payout user interface 410, such as at the one or more electronic displays 202 of the machines 20, though such is not required. The payout user interface 410 may be configured to provide multiple selectable payout options 421 for the total payout for the pay period.

The amount of the various payout options 321 may be determined based, at least in part, on analysis performed by a conversion module 413. For example, without limitation, the conversion module 413 may be configured to monitor exchange rates for currency, such as provided by one or more currency exchanges 415, stock market exchanges, other third-party systems reporting information regarding the same, combinations thereof, or the like. Such information may be updated periodically, continuously, intermittently, and/or in substantially real time. The displayed options 421 may be predetermined or customized.

The displayed options 421 may comprise a more options selectable area 423. Selection of the more options selectable area 423 may be configured to generate a prompt for the user to select from additional options, at least some of which may not already be displayed, including various publicly traded equities, certificates of deposit, bonds, currencies, gift cards, cash options, electronic transfer options, combinations thereof, or the like.

The displayed options 421 may comprise a split option selectable area 425. Selection of the split option selectable area 425 may be configured to generate a prompt for the user to split the total payout between multiple ones of the options 421, displayed or otherwise.

The payout user interface 410 may be configured to receive a user selection of one or more of the options 421, displayed or otherwise. Once a payout selection is made, the system 400 may be configured to execute the payout. The payment module 411 may be in electronic communication with various systems for executing the desired payout including, but not limited to, the exchange system 415, a banking system 417, a brokerage system 419, and an ATM 111.

In exemplary embodiments, the payout module 411 may alternatively or additionally be configured to record total payout and/or executed payment options using one or more blockchains 429. As each transaction is executed, a new block 427A, B, C may be generated comprising various datapoints or other criteria for the transaction including, but not limited to, the total payout, time and date of execution, the user selected payout options, executed price, quantity of items disbursed (e.g., number of shares, number of dollars, etc.), combinations thereof, or the like. Each new block 427A, B, C may comprise a hash of a prior block 427A, B, C and a new hash generated by the payout module 411, for example. The blockchain 429 may be stored at the database 100 in exemplary embodiments and updated with each transaction executed by the payout module 411. The blockchain 429 may be accessible, such as at an on-demand basis, by users of the machines 20 and data regarding the same may be displayed at the one or more electronic displays 202, for example, for user verification of the same.

Alternatively, or additionally, the blockchain 429 may be stored at one or more user devices 409. The user devices 409 may comprise smartphones, computers, tablets, servers, combinations thereof, or the like associated with the employee(s) earning the pay. The payout module 411 may be configured to transmit each new block 427A, B, C to at least the user devices 409 associated with the employee related to the payment, or to all user devices 409. The payout module 411 may be configured to only add the new block 427A, B, C to the blockchain 429 upon receipt of confirmation from the user devices 409 associated with the employee related to the payment and/or from at least a majority of the user devices 409. In this fashion, some or all of the user devices 409 may be used to authenticate newly added blocks 427A, B, C to improve security of the payment.

A separate blockchain 429 may be generated for each payout, for each machine 20, for each employee or other worker, combinations thereof, or the like. In other exemplary embodiments, a single blockchain 429 may be utilized.

Systems, methods, apparatuses, components, combinations thereof, or the like may be as shown and/or described in U.S. application Ser. No. 19/438,823 filed Jan. 2, 2026, the disclosures of which are hereby incorporated by reference, for any purpose, as if fully restated herein. For example, without limitation, the camera (item 18 therein, item 518 herein) and/or the augmented reality device (item 22 therein, item 522 herein) of the '823 application may form at least part of the data acquiring device 104, the additive manufacturing machine (item 12 therein, item 512 herein) of the '823 application may form at least part of the machine 20, the server(s) (item 16 therein, item 516 herein) of the '823 application may form part of the system 10, combinations thereof, or the like. Nevertheless, for the sake of providing a more complete application, certain contents of the '823 application are provided herein. Notably, item numbering from the '823 application is increased by 500 herewith to prevent overlap. Figure numbering is also updated for consistency.

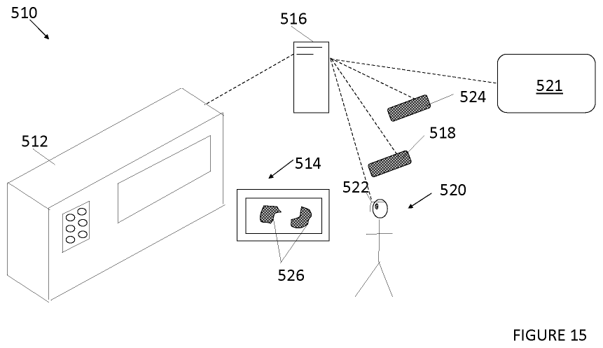

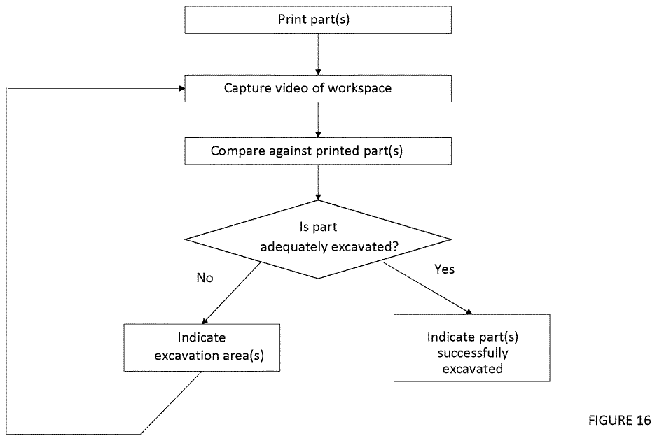

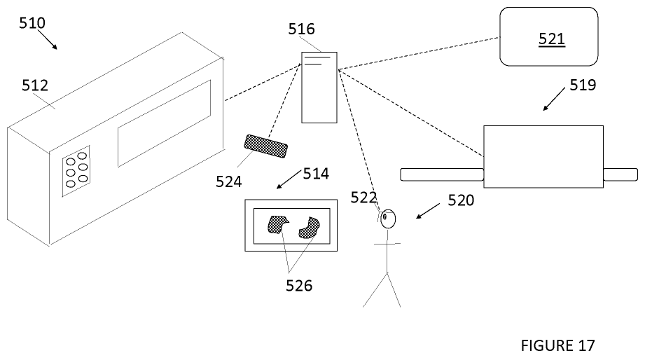

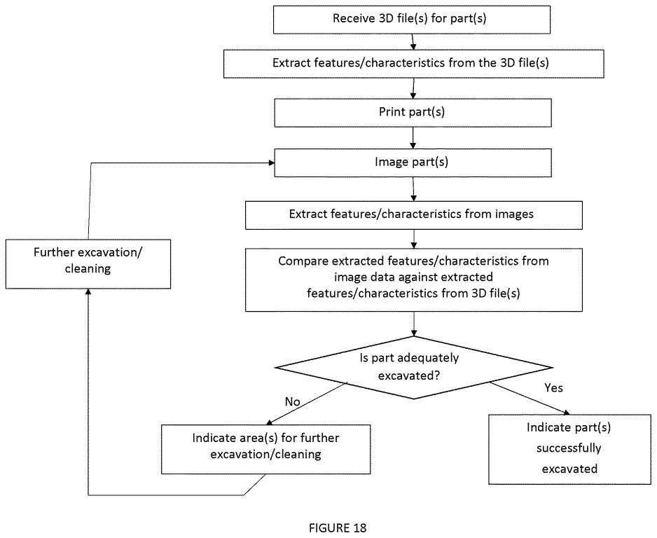

FIG. 15 and FIG. 17 illustrates exemplary systems 500 for indicating remaining build material for excavation and/or cleaning of parts 526 generated by additive manufacturing machines 512, such as 3D printing. FIG. 16 and FIG. 18 provide exemplary methods for operating the systems 500, or at least select components thereof.

Part(s) 526 are generated by an additive manufacturing machine 512 (“machine”), such as but not necessarily limited to a 3D printer, such as but not necessarily limited to a binder jetting device. Preferably, the machine 512 is one which utilizes sand or powder as the support material, though such is not necessarily required. For example, without limitation, the machine 512 may be one of various types and/or kinds of additive manufacturing machines 512 which utilized sand or powder beds.

The machine 512 may be configured to generate part(s) 526, such as at a build box 514. Preferably, but optionally, the build box 514 is removable, such as for cleaning/excavation and/or inspection and part 526 removal.

The manufactured parts 526 may be generated in accordance with 3D drawing data, such as uploaded to the machine 512 from one or more servers 516 and/or other remote electronic devices (e.g., smartphone, laptop, personal computer, tablet, combinations thereof, or the like). The machines 512 may be known additive manufacturing machines, such as but not limited to, those available from ExOne, and may operate, at least as to parts 526 manufacture, according to known techniques.

As illustrated with particular regard to FIGS. 15-16, a camera 518 at the associated workspace, preferably but optionally located outside of the machine 512, may capture images of the build box 514, such as after the parts 526 are generated and the build box 514 removed from the machine 512. A designated workspace area may be provided for capturing accurate images, such as an illuminated workbench or table area. In exemplary embodiments, the camera 518 is located to face downward onto the workbench or table area and additional lighting is provided adjacent to the camera 518 and oriented downward to face the workbench or table area. However, a variety of number and type of cameras 518 may be utilized, such as inside of and/or outside of the machine 512 and/or at various angles relative to the workspace and/or build box 514. Placing the camera(s) 518 outside of the machine may allow for more convenient excavation. Using multiple cameras 518 may allow more accurate data collection and excess build material identification. For example, without limitation, at least two offset cameras 518 may be utilized to capture depth information. The camera(s) 518 may be one or more known cameras and/or machine vision systems.

Alternatively, or additionally, images may be captured by an augmented reality (AR) device 522 worn by a technician 520, preferably the technician 520 performing the excavation and/or cleaning. The AR device 522 may include, for example without limitation, the HOLOLENS device available from Microsoft, the GLASS device available from Google, the VISION PRO device available from Apple, combinations thereof, or the like. Such AR devices 522 may comprise one or more embedded cameras for capturing such images, such as while the technician 520 is gazing at the build box 514.

As illustrated with particular regard to FIGS. 17-18, alternatively, or additionally, an X-ray machine 519 may be utilized to capture the images and/or image data. Advantageously, the use of an X-ray machine 519 may allow for at least limited penetration of remaining build material within a build box 514 for more accurate part(s) 526 imaging. The X-ray machine 519 may be one or more known X-ray machines 519, particularly but not exclusively, those utilized for industrial inspection. The X-ray machine 519 may optionally include or be associated with a conveyor for moving the build boxes 514 therethrough. The X-ray machine 519 may be sized to accommodate the same.

While the X-ray machine 519 is sometimes shown and/or described as being separate from the additive manufacturing machine 512, in other exemplary embodiments, the X-ray machine 519, or components thereof for providing the X-ray images and/or image data, may be provided within and/or as part of the additive manufacturing machine 512. This may be particularly advantageous, by way of example and without limitation, where the build box 514 is not otherwise removable.

The camera(s) 518, X-ray machine(s) 519, and/or AR devices 522 may herein be sometimes referred to generally as imaging devices.

Image data from the imaging devices (e.g., camera(s) 518, AR devices 522, and/or X-ray machines 519) may be transmitted to the same or different servers 516, such as by way of one or more wired and/or wireless connections. While server(s) 516 are sometimes shown and/or described, other computing devices may be utilized, such as personal computers, laptops, tablets, smartphones, combinations thereof, or the like.

The server(s) 516 may be configured to analyze the received image data, such as using one or more machine vision and/or artificial intelligence models (e.g., neural networks). For example, without limitation, the image data may be analyzed for various features and associated characteristics. Such features may include, by way of example and without limitation, part outlines, holes, protrusions, depressions, edges, surface texture, markings, combinations thereof, and the like. Such characteristics may include, by way of example and without limitation, sizes, locations, shapes, combinations thereof, and the like. Each of the parts may be associated with one or more features, and each of the features may be associated with one or more characteristics. Each of characteristics extracted from the image data of the part(s) 526 may be compared against expected characteristics. The comparison may be made on a part, feature, and characteristic specific basis. The expected characteristics and associated features may be specified in and/or extracted from the 3D print and/or part files for the part(s) 526. Such expected characteristics, features, and and/or part information may be supplied directly to the server(s) 516 by user devices and/or from the additive manufacturing machine 512, such as based on supplied information regarding part(s) 526 undergoing printing. The image data may be supplied to the server(s) 516 by the imaging devices.

The foregoing may be accomplished, at least in part, using feature extraction and/or machine vision techniques. In exemplary embodiments, without limitation, the server(s) 516 and/or AI model(s) are configured to compare the received image data, particularly the extracted features and characteristics thereof, against the 3D file data for the parts 526, such as to identify and compare the actual features and/or characteristics (e.g., size, shape, location) of the current excavation/cleaned parts 526 (from the image data) against corresponding expected features and/or characteristics (e.g., size, shape, location) (from the associated part 526 file(s)).

Where the server(s) 516 and/or AI model(s) determine that the feature characteristics of the current excavation/cleaned part(s) 526 (as determined from analysis of the image data) indicates that the part(s) 526 are outside of a predetermined margin (e.g., 5% or less, 1% or less, etc.) of the corresponding expected feature characteristic(s) (as determined from the part file), indication of one or more areas 528 where additional excavation and/or cleaning is required may be displayed at the AR device 522 and/or by way of the projection device(s) 524, and/or recorded, such as at the server(s) 516.

The predetermined margin(s) may be the same or different across parts 526 features, and/or characteristics thereof. Preferably, such indication and analysis is made on a feature by feature and characteristic by characteristic basis, however, a collective margin for multiple or all features and/or characteristics for a given part 526 may alternatively be utilized. Preferably, the predetermined margin is made on a percentage basis, however, absolute margins may be utilized. In exemplary embodiments, without limitation, the predetermined margins is/are set in the 3D part file data, such as acceptable tolerances for the part. However, in other exemplary embodiments, without limitation, the predetermined margin(s) is/are set by user preference, by default, combinations thereof, or the like.

Such analysis may be made by way of machine vision software, which may optionally utilize one or more artificial intelligence (“AI”) models, such as but not limited to, neural networks. For example, without limitation, the one or more servers 516 may comprise the machine vision software and/or AI model(s). The AI model(s), in exemplary embodiments, are trained using image data (e.g., from camera 518 images and/or X-ray 519 images) identified as being associated with sufficiently excavated/cleaned and non-sufficiently excavated/cleaned parts 526 and associated 3D files for such parts 526. Such images and/or image data may be manually tagged. The AI model(s) may optionally be updated over time, such as based on manually provided feedback regarding sufficiently and/or insufficiently excavated/cleaned parts 526. Such feedback may, for example and without limitation, be provided by technicians 520, supervisors, or the like, such as by way of associated electronic devices, by way of inventory control systems (e.g., based on customer rejections and/or returns), combinations thereof, or the like. The AI model(s) may be configured to utilize such feedback data to adjust the AI model(s) over time, such as part of training or retraining, thereby improving accuracy of results.

The analysis shown and/or described herein may optionally be completed using a digital twin of the parts 526, the build box 514, and/or manufacturing processes. The area(s) 528 for further excavation/cleaning may optionally be indicated in the digital twin.

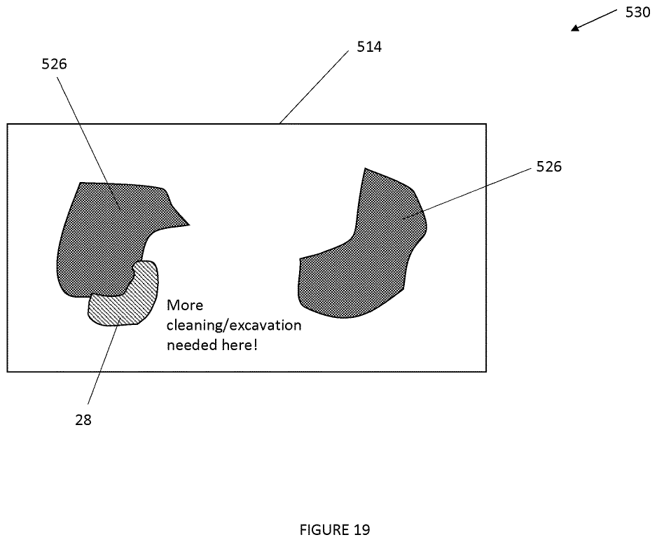

An exemplary output 30, by way of non-limiting example, is provided at FIG. 19. The output display 30 may be provided as a visual overlay at the AR device 522 and/or the projection device(s) 524 as an overlay at the build box 514. Alternatively, or additionally, such visual indications may be provided at one or more electronic displays 521 (e.g., TVs, display screens, smartphones, smartwatches, tablets, personal computers, laptops, combinations thereof, or the like), such as provided at the work area. The AR devices 522, projection device(s) 524, and/or electronic displays 521 may herein be sometimes referred to generally as visualization devices. Area(s) 528 for further excavation/cleaning may be indicated, such as by images, text, highlighting, texturing, outlining, color coding, pattern coding, combinations thereof, or the like at the visualization device(s). In this way, remaining build material, such as loose sand and/or support material, may be identified.

The electronic display(s) 521 may be configured to display a representation of the build box 514 with the area(s) 528 indicated at the representation, for example. The projection device(s) 524 may project the area(s) 528 directly on the build box 514, such as using projection mapping. The AR device(s) 522 may indicate the area(s) 528 when a wearer, such as the technician 520, is viewing the build box 514, such as based on the location of the build box 514, which may be identified by way of, for example and without limitation, machine vision (e.g., from imaging devices), a location device or other sensor embedded into the build box 514, combinations thereof, or the like.

Alternatively, or additionally, the server(s) 516 may be configured to cause the AR device 522 and/or projection device 524 to indicate the part(s) 526 as ideally formed in accordance with the 3D build files for display at the AR device 522 and/or overlay by way of the projection device(s) 524. Indication may include images, text, highlighting, outlining, color coding, combinations thereof, or the like. In this way, the technician 520 may be able to manually spot areas for further cleaning and/or excavation.

Where the server(s) 516 determines that the image data indicates that the part(s) 526 are within at least the predetermined margin(s) (e.g., 5% or less, 1% or less, etc.), such as for all features of the part(s) 526 of the build box 514, such as (but not limited to) in accordance with the margins specified in the part file, indication of a successfully excavated and/or cleaned part may be displayed at the AR device 522 and/or by way of the projection device(s) 524, and/or recorded, such as at the server(s) 516.

Where the server(s) 516 determines that the image data indicates that one or more of the part(s) 526 characteristics are not within at least the predetermined margin(s), such as (but not limited to) in accordance with the margins specified in the part file, the indications or the area(s) of attention (those not within the respective margin) may be identified at the AR device 522, by way of the projection device(s) 524, at the electronic display(s) 521, and/or otherwise recorded, such as at the server(s) 516. The technician 520 may perform further cleaning and/or excavation and then have the build box 514 and/or part(s) 526 thereof reinspected until an indication of successful cleaning/excavation is recorded.

This data may be traceable to the part(s) 526, build boxes 514, machines 512, and/or technicians 520, for example. Thereby providing an audit trail, certification, and/or history for generated parts 526.

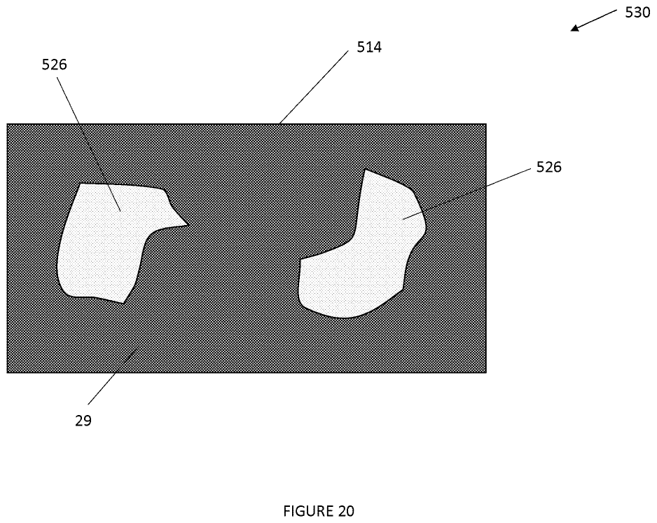

Alternatively, or additionally, as illustrated with particular regard to FIG. 20, the server(s) 516 may be configured to cause the AR device 522 and/or projection device 524 to indicate the support material 529 for the parts 526. In this way, the areas for excavation may be identified. In this way, the parts 526 may be relatively identified by inference (e.g., the areas not indicated as support material 529).

The parts 526, support material 529, and/or other areas for further cleaning/excavation may be identified using contrasting colors, textures, patterns, combinations thereof, or the like. A visual key or other identifier may be provided.

This may be provided for initial cleaning/excavation, further cleaning/excavation, and the like.

In an exemplary embodiments, without limitation, the systems and methods shown and/or described herein may be utilized in conjunction with a compensation tracking, visualization, and/or disbursement system, such as shown and/or described in U.S. Pat. No. 11,138,543 issued October 5, 520521, the disclosures of which are hereby incorporated by reference as if fully restated herein, for any purpose. By way of non-limiting example, a part 526, manufacturing run, and/or other unit of production may be recorded as being successfully completed only were the image data indicates that the extracted feature characteristics of the part(s) 526 are within the associated predetermined margins of the associated feature characteristics as expected (e.g., indicated and/or extracted from 3D part/print file), such as for all characteristics, features, and/or parts of the respective unit of production. This may be particularly advantageous when utilized with regard to pay-per-part compensation schemes and related systems, methods, and/or devices, such as by not limited as provided in the '543 Patent, in order to ensure quality while maintaining incentives towards efficient production.

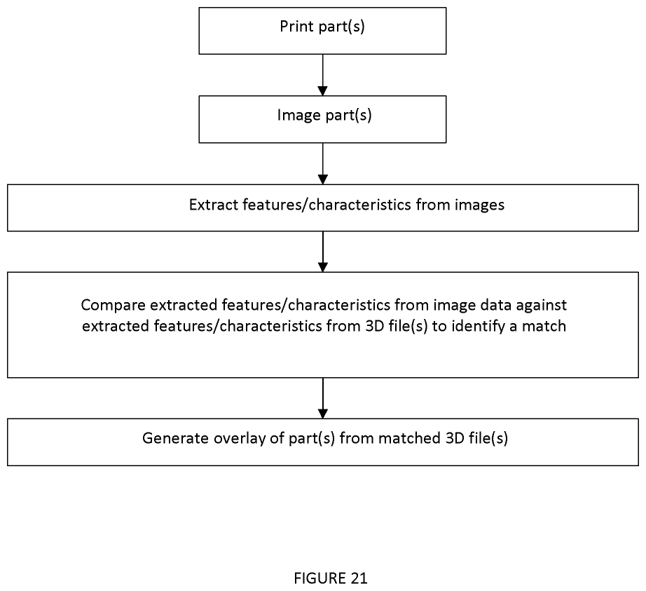

As illustrated with particular regard to FIG. 21, in exemplary embodiments, the part(s) 526 of the build box 514 may be matched with a corresponding part file, such as using the image data from the imaging devices (e.g., camera(s) 518, AR devices 522, and/or X-ray machines 519). The image data may be analyzed, such as using machine vision and/or feature extraction techniques (e.g., holes, shapes, outlines, surface features, combinations thereof, and the like), to identify features of the part(s) 526 and/or their locations. The extracted features information may be utilized to identify the part(s) 526. For example, without limitation, corresponding features information may be provided in 3D part files and/or extracted from the same. 3D part files describing parts at least within a predetermined tolerance (e.g., 5%) of the extracted features may be identified as a match. Completion of the corresponding part(s) 526 may be recorded. The corresponding part file data may thereafter be used to generate the overlay of the printed parts 526 and/or support material at the AR device 522, projection device(s) 524, and/or at electronic display(s) 521. In this way, an overlay of the printed part(s) 526 may be generated so that the team member can visualize and/or clean and/or excavate the part(s) 526. These overlays may be generated in alternative to, or in addition to, the overlays regarding areas needing further cleaning and/or excavation, for example. Some or all of the foregoing analysis and/or operations may be carried by out the server(s) 516, in exemplary embodiment.

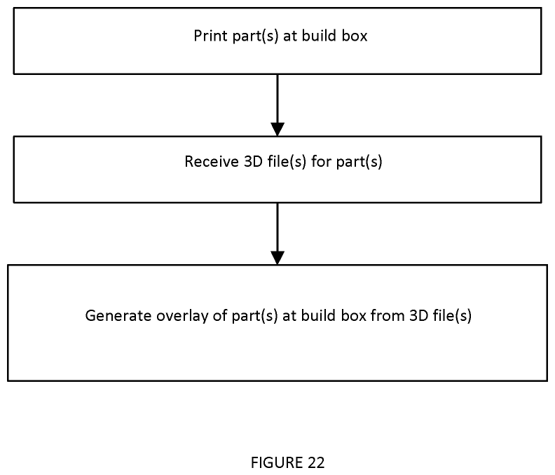

As illustrated with particular regard to FIG. 22, in exemplary embodiments, the 3D file(s) of the part(s) 526 of the build box 514 are provided, such as from the machine 512 and/or server(s) 516 based on operational data (e.g., what parts 526 are, or were most recently, printed by the machine 512). An overlay of the part(s) 526 is thereafter generated at the AR device 522, projection device(s) 524, and/or at electronic display(s) 521. In this way, an overlay of the printed part(s) 526 may be generated so that the team member can visualize and/or clean and/or excavate the part(s) 526. These overlays may be generated in alternative to, or in addition to, the overlays regarding areas needing further cleaning and/or excavation, for example. Some or all of the foregoing analysis and/or operations may be carried by out the server(s) 516, in exemplary embodiment.

In an exemplary embodiment, without limitation, a system for generating an overlay of parts at a build box generated by an additive manufacturing machine is provided, where said system comprises one or more non-transitory electronic storage devices comprising software instructions, which when executed, configure one or more processors to: receive 3D part files for the parts generated at the build box by the additive manufacturing machine; and cause an overlay of the parts to be generated at an augmented reality device or at the build box by a projection device.

Systems, methods, apparatuses, components, combinations thereof, or the like may be as shown and/or described in U.S. application Ser. No. 19/442,393 filed Jan. 7, 2026, the disclosures of which are hereby incorporated by reference, for any purpose, as if fully restated herein. For example, without limitation, team member 18 movement may be monitored and performance suggestions may be made as shown and/or described in the '393 application. Nevertheless, for the sake of providing a more complete application, certain contents of the '393 application are provided herein. Notably, item numbering from the '393 application is increased by 600 herewith to prevent overlap. Figure numbering is also updated for consistency.

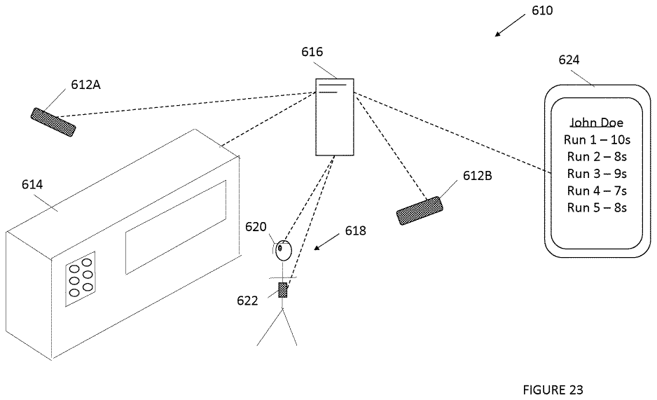

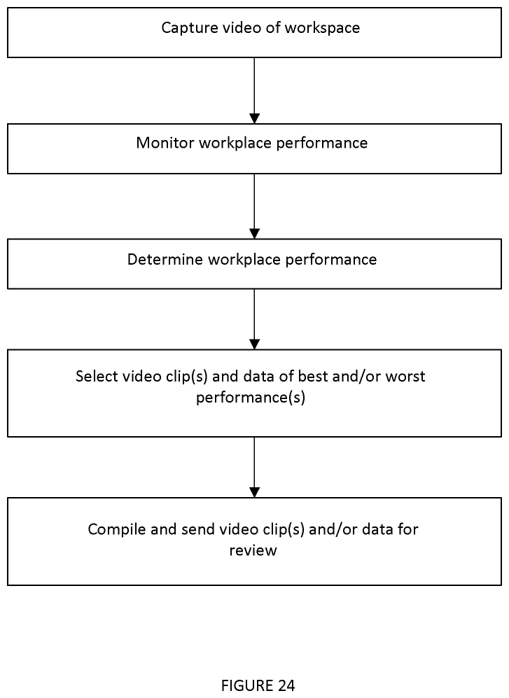

FIG. 23 illustrates an exemplary system 610 for monitoring and capturing workplace performance. FIG. 24 illustrates an exemplary method for operating the system 610. The system 610 may include one or more cameras 612. Multiple cameras 612A, 612B are preferably stationed to provide different viewing angles and/or positions of a given team member's 618 workspace. This may provide multiple perspectives and/or depth information, which may be particularly useful when working around large equipment 614, facing in different directions during operations, and/or for a user to fully understand his/her position in three-dimensional space. One or more items of equipment 614 may be provided within the team member's 618 workspace.

The items of equipment 614 may include any type or kind of industrial equipment, such as additive manufacturing machines, subtractive manufacturing machines, fabrication equipment (manual and/or automated, e.g., lathes, welding equipment, bending machines, drill presses, etc.), CNC machines, forging equipment, extrusion equipment, chemical processing equipment, excavation equipment, industrial robots (e.g., for material handling, welding, inspection, assembly, and the like), co-bots, combinations thereof, or the like, by way of example and without limitation. In other exemplary embodiments, non-manufacturing types or kinds of equipment may be provided, such as but not limited to, computers, pharmaceutical packaging equipment, pick and packaging equipment, warehouse storage equipment, logistics equipment, healthcare equipment, combinations thereof, or the like, by way of example and without limitation. Various types and/or kinds of equipment from a wide variety of fields may be utilized, preferably the type and/or kind which require manual, human movements to accomplish various commercial, production activities, weather to produce a good, provide a service, and/or accomplish some kind of type or kind of task.

The team members 618 may each be provided with one or more AR devices 620 and/or biomechanics monitoring devices 622 for wearing, at least while participating in production activities. The biomechanics monitoring devices 622 may include, for example without imitation, location tracking devices, movement tracking devices, range of motion sensors, accelerometers, GPS devices, gyroscopes, combinations thereof, or the like. These may be provided as part of and/or within one or more housings and/or as part of devices configured to be worn in various places, such as but not limited to, the team member's 618 hand, arm, shoulder, chest, torso, belt line, waistline, leg, foot, ankle, head, combinations thereof, or the like.

The AR devices 620 may include, for example without imitation, GLASS available from Google, HOLO LENS available from Microsoft, combinations thereof, or the like. The AR devices 620 may be provided in addition to, or in substitution of, the biomechanics monitoring devices 622. For example, without limitation, the AR devices 620 may provide location and/or movement tracking.

Data from some or all of the camera(s) 612, equipment 614, AR device(s) 620, and biomechanics monitoring device(s) 622 for a given team member 618 and/or workplace, and/or multiple team members 618 and/or workspaces may be transmitted to one or more servers 616 for storage and/or processing. The server(s) 616 may be remote from the workspace, though such is not necessarily required.

The server(s) 616 may be in electronic communication with one or more electronic devices 624. The electronic device(s) 624 may include, for example without limitation, smartphone, tablets, laptops, computers, electronic displays, combinations thereof, or the like. The electronic device(s) 624 may be local to the workspaces, though such is not necessarily required.

Video of the workspace may be captured by the camera(s) 612 and/or AR device(s) 620 and transmitted to the server(s) 616 for electronic storage. Other data regarding workplace performance may be gathered, such as from the equipment 614, AR device(s) 620, and/or biomechanics monitoring device(s) 622, and may be transmitted to the server(s) 616 for electronic storage.

The video and/or other data may be provided with time information recorded prior to transmission and/or may be associated with a current time at the server(s) 616, such as upon receipt. This may provide central coordination of time. Alternatively, or additionally, the server(s) 616 may be configured to periodically coordinate time across the various system 610 components, such as by regularly signaling and/or clock updates. The video and/or other data is preferably stored in a time correlated format.

The video and/or other data may be processed by the server(s) 616, such as into various summaries, time dependent graphical displays, combinations thereof, or the like. These displays may be presented at some or all of the electronic devices 624, by way of example. Workplace performance of team members 618 may be monitored, such as through the aforementioned displays, on an individual team member 618 basis and/or on a group basis for some or all team members 618 and/or workspaces.

Team member 618 performance may be determined, such as on an individual team member 618 basis. In exemplary embodiments, without limitation, a time may be kept between each unit of output for the team member 618. The initiation and/or completion of each unit of production may be determined based on data from one or more of the equipment 614, the AR devices 620, and/or the cameras 612. For example, without limitation, the equipment 614 may be configured to record when each unit of production is initiated and completed. Alternatively, or additionally, images captures from the AR devices 620 and/or cameras 612 and processed by machine vision technology and may be configured to determine when a unit of production is initiated and/or completed.

In exemplary embodiments, without limitation, data from the equipment 614 is utilized to determine when each unit of production is initiated and completed, such as where manufacturing cycles for units of production are completed. Optionally, machine vision processed images from the AR device(s) 620 and/or camera(s) 612 is utilized to determine if the unit of production meets certain acceptability requirements. If so, the unit may be recorded as completed. If not, the time may be non-recorded, deleted, recorded as incomplete, recorded as scrap, cumulated until an acceptable unit is completed, combinations thereof, or the like.

By way of non-limiting example, in a manufacturing environment, such as but not limited to an additive manufacturing environment, the equipment 614 (e.g., a binder-jetting printer) may provide data to the server(s) 616 regarding printing run initiation and completion (e.g., a signal that the printer has started a production run and/or a start time). Time may be locally recorded at the equipment 614 and/or at the server(s) 616.

In other exemplary embodiments, unit completion is electronically recorded by user input or other user indication (e.g., user presses button to indicate unit complete).

For each completed part, data from the AR device(s) 620 of the team member 618 and/or the camera(s) 612 may be analyzed, such as by machine vision technology, to compare the completed part against data for a desired output. In the exemplary embodiment, without limitation, of an additive manufacturing environment, images from the AR device(s) 620 and/or the camera(s) 612 may include images of the part(s) from a printing run post excavation (manual or otherwise). The data for the desired output may include, by way of example without limitation, electronic drawing files, extracted feature data of a completed exemplary part (e.g., by 3D scanning), 3D file data (e.g., part file, print file), combinations thereof, or the like.

Where the part is within an acceptable, predetermined margin of error from the data for the desired output, the part may be recorded as successfully completed. The time to complete the production item may be recorded based on the start and initiation data from the equipment 614 or other system 610 components, such as in association with data identifying the team member 618, part, workspace, team, coach, combinations thereof, or the like. Alternatively, or additionally, adequacy of the production item (part or otherwise) may be determined manually, such as by the team member 618 and/or another team member 618 and recorded, such as by manual entry as such.

The start and end data and associated times may be determined based on data from various system 610 components. For example, without limitation, in the additive manufacturing environment example, the start time may be recorded based on data from the equipment 614 (e.g., 3D printer) while the end time may be recorded based on data from the AR device(s) 620 and/or camera(s) 612 identifying an acceptable part, such as post excavation.

The data from some or all of the various system 610 components may be electronically stored at the server(s) 616. The server(s) 616 may be configured to present some or all of the data, such as at the electronic devices 624, in various forms, such as part of various graphical displays, such as in summary form, for each team member 618, group, coach, production unit type, day, time frame, item of equipment 614, combinations thereof, or the like. The data may be presented in various formats, such as but not limited to, summary style, time dependent format, ranking format (e.g., for individual team member 618, against other team members 618, for the item of equipment 614, for the type of unit being produced, etc.), combinations thereof, or the like.

For example, without limitation, team members 618 and/or coaches may remotely view the video and/or other data at their respective electronic devices 624. Alternatively, or additionally, a central display and/or ranking of certain of the video and/or other data may be provided at an electronic device 624 (e.g., display at the workplace) to competitively motivate team members 618.

The server(s) 616 may be configured to, automatically and/or on demand, retrieve and/or select video clips and/or other data regarding various team member 618 performances for transmission to the remote devices 624. For example, without limitation, the server(s) 616 may be configured to retrieve and/or select video clips from some or all of the camera(s) 612 of the workspace for a team member's 618 best and/or worst performances, such as based on time to complete a unit of production, scrap rate, combinations thereof, or the like. Those video clips may be complied and transmitted to some or all of the electronic devices 624 for viewing. Alternatively, or additionally, other data may be included, such as position information from the biomechanics monitoring devices 622.

In exemplary embodiments, without limitation, the data from the camera(s) 612, biomechanics monitoring devices 622, and/or AR devices 620 may be utilized to create a simulation of the team member's 618 position in space over time, thereby generating a simulation of the team member's 618 movements. In this way, the team member's 618 performance may be reviewed, such as by the team member 618, coach, other team member 618, combinations thereof, or the like. This may allow the same or different movements to be implemented by the team member 618 or other personnel, such as in future production operations. This may allow lessons learned to be implemented over time to improve efficiency, among other advantages. For example, without limitation, a top predetermined number (e.g., 3, 5, 610, etc.) of best and/or worst production performances over a predetermined period of time (e.g., daily, weekly, etc.) may be automatically ranked, retrieved, and/or complied into a highlight reel which is sent to the electronic device 624 associated with the team member 618 and/or coach.

To provide another exemplary embodiment, without limitation, in a pharmaceutical dispensing operation, team members 618 may include pharmacy technicians and pharmacists. As technician team members 618 prepare prescriptions using equipment 614, such as workstations, pill counters, optical scanning devices, combinations thereof, or the like, which are reviewed by pharmacist team members 618, time to completion for each prescription for dispensing may be recorded. The video clips and/or other data may be recorded for subsequent review. The team members 618 may identify movements that resulted in increased or decreased efficiency.

While a single workspace, team member 618, item of equipment 614, or the like is sometimes shown and/or described, the camera(s) 612 may view multiple workspaces and/or monitoring across multiple workspaces, team members 618, items of equipment 614, combinations thereof, and the like. For example, without limitation, a whole factory or factory floor may be so monitored for production across multiple items of equipment 614 and/or team members 618. Data for each team member 618 and/or item of equipment 614 may be analyzed separately or together in such embodiments.

Alternatively, or additionally, where multiple items of equipment 614 and/or team members 618 utilized as part of generating a unit of production, performance may be monitored across the various items of equipment 614 and/or team member 618 such that each completion time is associated with multiple team members 618 for example. Relevant video clips and/or other data may be selected, retrieved, complied, and/or transmitted accordingly such that only specific team member(s) 618 and/or equipment 614, or all team members 618 and/or equipment 614 may be viewed.

Time data from the equipment 614, the camera(s) 612, AR device(s) 620, and/or biomechanics monitoring device(s) 622 may each be time dependent, preferably as tracked and recorded by the one or more servers 616 to provide a consistent time measurement, and cross referenced to build a database of information from which to generate the video clips and/or other data.

The server(s) 616 may be configured to communicate with the various system 610 components on a closed, preferably encrypted, network, though such is not necessarily required. The server(s) 616 may be configured to convert the data received from some or all of the system 610 components into a common format for storage. Alternatively, or additionally, the server(s) 616 may be configured to record start and end times and/or other data in a common format based on the data in various formats from the various system 610 components. This may permit and enhance interoperability and/or security.

The system 610 may utilize some or all of what is shown and/or described in US Pub. No. 2021/0357845 published November 618, 62021, from application Ser. No. 17/387,6180 filed July 28, 62021, the disclosures of which are hereby incorporated by reference as if fully restated herein for any purpose.

The units of production shown and/or described herein do not necessarily need to be completed items of production. For example, the units may be parts which are subsequently processed by the same or another team member 618 and/or items of equipment 614, assembled into a larger product, be portions of a service item, combinations thereof, or the like.

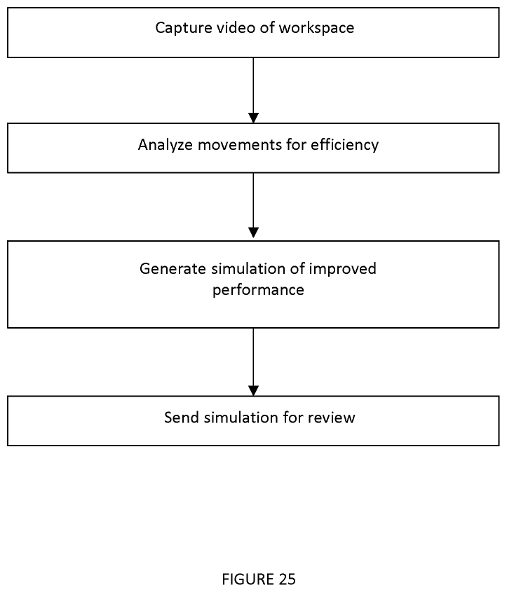

As illustrated with particular regard to FIG. 25, in other exemplary embodiments, video of the workspace may be analyzed, such as at the sever(s) 616, for efficiency. In exemplary embodiments, without limitation, the video files and/or other data (e.g., from the biomechanics monitoring devices 622, AR devices 620) are analyzed using artificial intelligence (AI) models.

Initially, video files, if utilized, may be analyzed using machine vision software to identify the team member 618 in the video and/or their body outline and/or movements. The machine vision software may be configured to analyze the video files in conjunction with data from the biomechanics monitoring device(s) 622 and/or AR device(s) 620 for the team member 618 such as to more accurately determine location of the team member 618.

Alternatively, or additionally, the machine vision software may be trained on an outline of the team member 618 and/or biomechanics monitoring device(s) 622 and/or AR device(s) 620 to identify the same more accurately in videos. Such data may be manually tagged.

Alternatively, or additionally, the biomechanics monitoring device(s) 622 and/or AR device(s) 620 may comprise optically scannable objects such as target points, QR codes, barcodes, or other optically identifiable objects for identification by the machine vision software. The machine vision software may be trained on such objects for more accurate position identification. Each of the objects may be coded by location. For example, without limitation, the team member 618 may wear wrist band, ankle bands, and/or forehead bands with printed codes or symbols which are readily identifiable by the machine vision software. The objects may be the same or different at some or all locations, such as for increased accuracy of position identification (e.g., object 1 for left wrist, object 2 for right wrist, etc.) and analysis.

The AI model(s) may be configured to compare the determined location of the team member 618 against data for other team members 618 and/or historical data for the team member 618. Movements associated with relatively high efficiency may be selected as targets, and neural networks may be employed, by way of example, to compare the team member's 618 current movements against the training data for efficiency. The AI model(s) may identify movements which result in decreased efficiency, such as measured in time to generate the units of production.

The server(s) 616 may be configured to generate a simulation of team member 618 movements resulting in improved efficiency movements. The simulation may be generated and transmitted to the electronic device(s) 624 for review. For example, without limitation, the AI model(s) may be configured to analyze movements against training data of historical movements for discrepancies resulting in decreased efficiency, such as based on time to complete units of production. The server(s) 616 may be configured to generate simulations, such as in the form of an avatar for the team member 618 generating the units of production, with movements perceived by the AI model(s) to improve efficiency. The avatar may take on various forms, such as but not limited to a stick figure type outline.

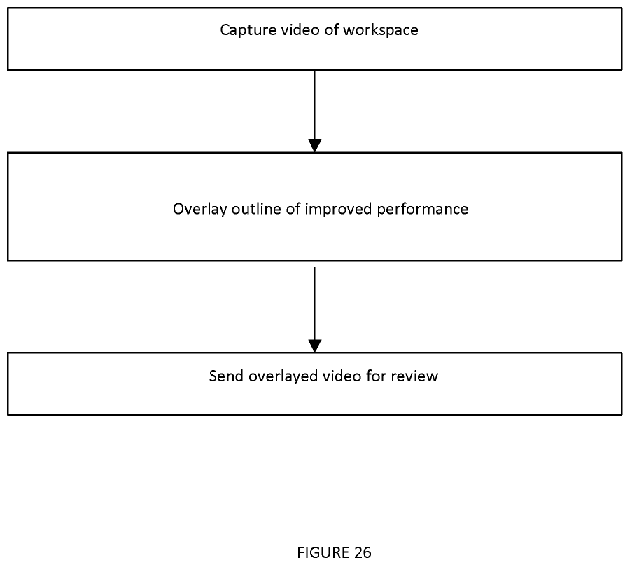

Alternatively, or additionally, as illustrated with particular regard to FIG. 26, the server(s) 616 may be configured to generate annotated versions of the video files may with feedback regarding team member 618 performance and areas of potential improvement identified by the AI model(s). The annotated video files may be transmitted to the electronic device(s) 624 for review. Such annotation may take the form of an overlay of a video of the team member 618 with the simulation and/or an overlay of video from a more efficient performance. Alternatively, or additionally, the annotation may take the form of inserted written (e.g., text, symbol, etc.) and/or verbal feedback.

The AI model(s) may, in exemplary embodiments, be trained with various human biomechanics data, such as ergonomic movements, idealized movements, acceptable movement parameters, regulatory parameters (e.g., OHSA guidelines or regulations), combinations thereof or the like, such as forming part of predefined limits and/or idealized guidelines. This may be used to suggest user movements and/or identify (e.g., for annotation) user movements for improvement.

In other exemplary embodiments, the AI model(s) are not necessarily required insofar as the overlay or simulation may be provided based on historical video files or data, such as in video form, simulation, and/or avatar. This may allow a team member 618 to essentially “ghost race” against historical or simulated performance data.

While an overlay is sometimes shown and/or discussed, other formats may be utilized, such as side-by-side, picture-in-picture, combinations thereof, or the like.

While the overlays or displays are sometimes shown and/or described herein as being presented after a run is complete, for example, in other exemplary embodiments, the overlays or displays may be generated in at least substantially real-time (e.g., accounting for normal delays in processing, transmission, and the like). In this way, the team member 618 may be provided with real time feedback, such as by way of highlighting or other annotation, such as indicating actual movements of reduced efficiency and/or movements that would improve efficiency. In this way, for example and without limitation, the team member's 618 current, actual work may be compared against a high efficiency manufacturing run (e.g., prior highest efficiency record) and/or a theoretical best run, such as in at least substantially real time, and annotations/highlights may be provided which demonstrate areas for improvement.

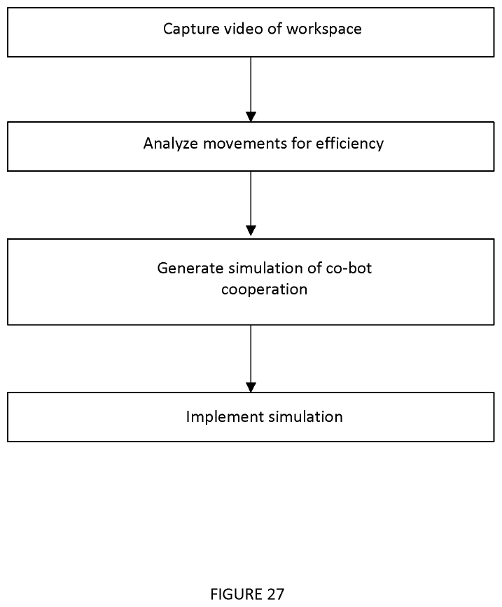

Alternatively, or additionally, as illustrated with particular regard to FIG. 27, the server(s) 616 may be configured to utilize user movement data to determine cooperative equipment 614 operation, such as using the AI model(s) and one or more predefined prompts regarding input data and desired results. This may take the form of, for example without limitation, equipment 614 cycle operations timed to optimize user movements (e.g., shorter cycles where team members idle, longer cycles were team members struggling to keep up), equipment 614 movements (e.g., co-bots to improve efficiency, reduce undesired movements), combinations thereof, or the like. The server(s) 616 may be configured to suggest changes for manual adopting, or may automatically implement changes. The server(s) 616, particularly where using the AI model(s), may continue to receive the data shown and/or described herein to understand if the implemented changes resulted in improved times and train or retrain the AI model(s) accordingly.

Certain exemplary embodiments are provided herein, by way of example and without limitation.

Embodiment 1—A computerized method for providing real-time earnings feedback to a team member performing a production activity, the method implemented by one or more processors, said method comprising: acquiring production data for a production activity from a machine for performing the production activity to generate units of output, said production data acquired on an ongoing basis while the production activity is underway; generating, on an ongoing basis while performance of the production activity is underway, an evaluated earnings rate for said team member by multiplying the production data with a unit of output based earnings rate for said team member; transmitting the evaluated earnings rate to a wearable electronic device secured to the body of said team member; and displaying and updating, at the wearable electronic device, a graphic representing the evaluated earnings rate, wherein the graphic is automatically selected from a plurality of graphics each associated with a threshold evaluated earnings rate, the selected graphic being associated with the highest surpassed threshold based on the most recently determined evaluated earnings rate.

Embodiment 2—The method of embodiment 1 wherein: the wearable electronic device comprises a watch-style device adapted to secure around a wrist of said team member.

Embodiment 3—The method of embodiment 1 wherein: the wearable electronic device comprises an augmented reality headset worn by said team member; and the graphic representing the evaluated earnings rate is displayed as an overlay on a view of the team member's workspace rendered by the augmented reality headset, said overlay positioned within the team member's field of view during performance of the production activity.

Embodiment 4—The method of embodiment 1 further comprising: simultaneously transmitting the evaluated earnings rate to a plurality of electronic devices associated with said team member, said plurality of electronic devices comprising at least two of: an electronic display at the machine, a wearable electronic device secured to the body of the team member, and a mobile device of the team member; and displaying and updating the graphic representing the evaluated earnings rate substantially simultaneously across each of said plurality of electronic devices.

Embodiment 5—The method of embodiment 1 further comprising: maintaining, in the database, a record of a personal best evaluated earnings rate for said team member over a predetermined historical period; comparing the most recently determined evaluated earnings rate against the personal best evaluated earnings rate; and in response to the most recently determined evaluated earnings rate exceeding the personal best evaluated earnings rate, displaying a milestone achievement notification at the electronic display and updating the personal best evaluated earnings rate stored in the database.

Embodiment 6—The method of embodiment 5 further comprising: tracking a consecutive-period streak count reflecting a number of successive pay periods during which said team member's evaluated earnings rate exceeded a predetermined earnings rate target; displaying the consecutive-period streak count at the electronic display during performance of the production activity; and in response to the consecutive-period streak count reaching a streak threshold, displaying a streak achievement notification at the electronic display.

Embodiment 7—The method of embodiment 1 further comprising: calculating, on an ongoing basis while the production activity is underway, a projected total earnings amount for a current pay period based on the most recently determined evaluated earnings rate and a remaining time in the current pay period; and displaying the projected total earnings amount at the electronic display simultaneously with the graphic representing the evaluated earnings rate.

Embodiment 8—The method of embodiment 7 further comprising: receiving, at the one or more processors, one or more investment parameters including at least an investment return rate and an investment duration; calculating a projected investment value of the projected total earnings amount after the investment duration at the investment return rate; and displaying the projected investment value at the electronic display alongside the projected total earnings amount.

Embodiment 9—A computerized method for providing real-time earnings feedback to a worker performing a service activity, the method implemented by one or more processors, said method comprising: acquiring service performance data for a service activity performed by a worker, said service performance data comprising a count of service units completed by the worker, said service performance data acquired on an ongoing basis while the service activity is underway by way of a data acquiring device associated with the service activity; retrieving a unit of service based earnings rate for the worker from a database; generating, on an ongoing basis while the service activity is underway, an evaluated earnings rate for said worker by multiplying the service performance data with the unit of service based earnings rate; and displaying and updating, on an ongoing basis, a graphic at an electronic display associated with the worker, said graphic representing the evaluated earnings rate and automatically selected from a plurality of graphics each associated with a threshold evaluated earnings rate.

Embodiment 10—The method of embodiment 9 wherein: the service activity comprises a healthcare service activity; the service units comprise at least one of patient visits completed, procedures performed, prescriptions dispensed, or assessments documented; and the data acquiring device comprises an electronic health record system, patient scheduling system, or automated dispensing system in electronic communication with the one or more processors.

Embodiment 11—The method of embodiment 9 wherein: the service activity comprises a logistics or delivery activity; the service units comprise at least one of packages scanned, deliveries completed, routes finished, or pick-and-place operations performed; and the data acquiring device comprises at least one of a barcode scanner, RFID reader, GPS tracking device, or warehouse management system in electronic communication with the one or more processors

Embodiment 12—The method of embodiment 9 wherein: the service activity comprises a food service or restaurant activity; the service units comprise at least one of orders fulfilled, meals prepared, tables served, or transactions completed; and the data acquiring device comprises a point-of-sale system, kitchen display system, or order management system in electronic communication with the one or more processors.

Embodiment 13—A computerized method for providing real-time earnings feedback to a gig worker performing on-demand work activities, the method implemented by one or more processors, said method comprising: receiving, at the one or more processors, work completion data from a gig platform for work activities completed by the gig worker during an active work session, said work completion data acquired on an ongoing basis while the active work session is underway; retrieving a per-unit earnings rate for the gig worker from the gig platform or a database; generating, on an ongoing basis during the active work session, an evaluated earnings rate for the gig worker by multiplying the work completion data with the per-unit earnings rate; displaying and updating, on an ongoing basis at an electronic device of the gig worker, a graphic representing the evaluated earnings rate; and automatically selecting the graphic from a plurality of graphics each associated with a threshold evaluated earnings rate, the selected graphic associated with the highest surpassed threshold based on the most recently determined evaluated earnings rate.

Embodiment 14—The method of embodiment 13 further comprising: receiving, at the one or more processors on an ongoing basis, a demand multiplier from the gig platform reflecting a current demand condition for the work activity; adjusting the per-unit earnings rate by the demand multiplier to generate an adjusted per-unit earnings rate; recalculating the evaluated earnings rate using the adjusted per-unit earnings rate; and displaying the adjusted evaluated earnings rate and the demand multiplier simultaneously at the electronic device.

Embodiment 15—The method of embodiment 13 further comprising: receiving work completion data from a plurality of gig platforms for work activities completed by the gig worker during the active work session; generating a combined evaluated earnings rate aggregating the evaluated earnings rates across each of said plurality of gig platforms; and displaying and updating the combined evaluated earnings rate at the electronic device.

Embodiment 16—A computerized method for coaching a team member to increase an evaluated earnings rate during a production activity, the method implemented by one or more processors, said method comprising: acquiring production data for a production activity from a machine on an ongoing basis while the production activity is underway; generating, on an ongoing basis, an evaluated earnings rate for a team member performing the production activity by multiplying the production data with a unit of output based earnings rate for the team member; analyzing the evaluated earnings rate using an artificial intelligence model trained on historical production data and historical evaluated earnings rates for the team member or a plurality of team members to identify production patterns associated with higher evaluated earnings rates; generating, based on said analysis, one or more coaching recommendations for the team member directed to increasing the evaluated earnings rate; and transmitting the one or more coaching recommendations to an electronic device of the team member.

Embodiment 17—The method of embodiment 16 further comprising: displaying the one or more coaching recommendations at the electronic device simultaneously with the graphic representing the evaluated earnings rate such that the coaching recommendations are presented to the team member in the context of the current earnings rate; and updating the one or more coaching recommendations on an ongoing basis during the production activity as the evaluated earnings rate changes.

Embodiment 18—The method of embodiment 16 further comprising: analyzing, using the artificial intelligence model, historical production data for said team member to identify an earnings rate ceiling representing the team member's historical maximum sustained evaluated earnings rate; generating a visual indicator of the earnings rate ceiling at the electronic display showing a threshold between the team member's current evaluated earnings rate and the earnings rate ceiling; and in response to the most recently determined evaluated earnings rate approaching the earnings rate ceiling within a predetermined margin, displaying a breakthrough notification at the electronic display prompting the team member to sustain or increase output to surpass the earnings rate ceiling.

Embodiment 19—The method of embodiment 16 further comprising: acquiring production data and generating evaluated earnings rates for a plurality of team members performing the production activity; analyzing the evaluated earnings rates for said plurality of team members using the artificial intelligence model to identify team-level production patterns associated with higher aggregate evaluated earnings rates; generating team-level coaching recommendations directed to increasing aggregate evaluated earnings rates across the plurality of team members; and transmitting the team-level coaching recommendations to an electronic device of a supervisor or manager associated with the plurality of team members.

Embodiment 20—A computerized method for providing real-time visual earnings feedback to a team member performing a production activity, said method implemented by one or more processors in electronic communication with a machine performing the production activity, said method comprising: acquiring production data for a production activity from the machine for performing the production activity to generate units of output, said production data acquired on an ongoing basis while the production activity is underway by way of a data acquiring device forming part of, or in electronic association with, the machine; retrieving a unit of output based earnings rate data for the team member tasked with performing the production activity with the machine from a database; generating, on an ongoing basis while performance of the production activity is underway, an evaluated earnings rate for said team member by at least one of the one or more processors multiplying the production data with the output based earnings rate for said team member, said processor being in electronic communication with the data acquiring device and a machine-integrated electronic display communicatively coupled to the data acquiring device; and displaying and updating, on an ongoing basis and in real-time while performance of the production activity is underway, a graphic at the machine-integrated electronic display, said graphic representing the evaluated earnings rate and rendered in real-time responsive to the evaluated earnings rate crossing one of a plurality of threshold evaluated earning rates, wherein the graphic is automatically selected from a plurality of graphics, each of said plurality of graphics being associated with one of the threshold evaluated earnings rates, the selected graphic being the graphic of the plurality of candidate graphics associated with a highest surpassed one of the threshold evaluated earning rates based on a most recently determined evaluated earnings rate.

Embodiment 21—The method of embodiment 20 wherein: the data acquiring device is a programmable logic controller of the machine for controlling, at least in part, performance of the production activity.

Embodiment 22—The method of embodiment 20 wherein: the data acquiring device comprises a camera for capturing image data of units of output generated by the machine during performance of the production activity.

Embodiment 23—The method of embodiment 22 wherein: the camera forms part of an augmented reality device.

Embodiment 24—The method of embodiment 23 further comprising: generating the production data at least in part by comparing the captured image data of the units of output against a build file for the units of output.

Embodiment 25—The method of embodiment 24 wherein: the machine comprises an additive manufacturing machine.

Embodiment 26—The method of embodiment 25 wherein: the additive manufacturing machine comprises a binder jetting machine.

Embodiment 27—The method of embodiment 26 wherein: the electronic display is a user interface for the binder jetting machine, part of the augmented reality device, or a separate electronic display.

Embodiment 28—The method of embodiment 20 further comprising: determining a total amount of pay for a pay period of working time upon completion of the pay period of working time by multiplying the acquired actual production data for the pay period of working time with the earnings data for said team member; converting the total amount of pay for the pay period of working time into a plurality of payment options; displaying each of the plurality of payment options at the electronic display; receiving a user selection of at least one of the plurality of payment options; and causing execution of an electronic transaction in accordance with said user selection.

Embodiment 29—The method of embodiment 28 further comprising: generating and storing a blockchain reflecting transaction data for each of said electronic transactions, including generating a new block of said blockchain upon completion of each of said electronic transactions, wherein each new block stores the evaluated earnings rate at the time of said electronic transaction and the selected graphic associated therewith, and electronically transferring each new block to a database for storage, said new block being authenticated by at least one user device in electronic communication with the database.

Embodiment 30—The method of embodiment 28 wherein: the plurality of payment options are selected comprise cash, electronic banking transfer, alternative currency, and stock purchase.

Embodiment 31—The method of embodiment 20 further comprising: acquiring additional production data for the production activity from additional ones of the machine for performing the production activity to generate units of output, said additional production data acquired on an ongoing basis while the production activity is underway by way of data acquiring devices forming part of, or in electronic associated with, the additional ones of the machine; retrieving unit of output based earnings rate data for additional team members tasked with performing the production activity with the additional ones of the machine from the database; generating, on an ongoing basis while performance of the production activity is underway, evaluated earnings rates for the additional team members by multiplying the additional production data with the output based earnings rate, said evaluated earnings rates being generated on a machine specific basis for each of the additional ones of the machine; generating a ranking of said team member and each of the additional team members based on the evaluated earnings rate for the team member and the evaluated earnings rates for the additional team members; and causing the ranking to be displayed at the electronic display or another electronic display.

Embodiment 32—The method of embodiment 31 further comprising: aggregating the evaluated earnings rates for said team member and said additional team members in real time to generate an aggregate evaluated earnings rate for a production floor, shift, department, or facility; generating a supervisor dashboard at a supervisor electronic display comprising the aggregate evaluated earnings rate, individual evaluated earnings rates for each of said team member and said additional team members, and a ranking of said team member and said additional team members based on their respective evaluated earnings rates; and updating the supervisor dashboard on an ongoing basis while the production activity is underway.

Embodiment 33—The method of embodiment 31 wherein: the ranking includes, for each of the additional team members, a respective graphic of the plurality of graphics selected based on the evaluated earnings rates for the additional team members and the threshold evaluated earning rates.

Embodiment 34—The method of embodiment 31 wherein: the ranking includes graphical objects, each representing one of the team members and each of the additional team members.

Embodiment 35—The method of embodiment 34 wherein: the ranking includes simulated racetrack; and each of the graphical objects comprises a simulated racehorse moving about the simulated racetrack.

Embodiment 36—A system for increasing engagement in a production activity, said system comprising: one or more non-transitory electronic storage devices storing software instructions, which when executed, configure one or more processors to perform the method of embodiment 20.

Embodiment 37—The system of embodiment 36 further comprising: the one or more processors; the machine; the data acquiring device; the database; and the electronic display.

Embodiment 38—The system of embodiment 37 further comprising: generating the production data at least in part by comparing captured image data of the units of output against a build file for the units of output, the captured image data being captured by a camera of the data acquiring device forming part of an augmented reality device during performance of the production activity, and wherein the machine comprises a binder jetting machine operated at least in part using the build file.

Embodiment 39—The method of embodiment 20 further comprising: receiving, at the one or more processors, a production demand signal indicating a current production demand level for units of output; adjusting the unit of output based earnings rate for said team member dynamically based on the production demand signal to generate an adjusted earnings rate; and recalculating the evaluated earnings rate using the adjusted earnings rate and displaying the updated graphic reflecting the adjusted evaluated earnings rate at the electronic display.

Embodiment 40—The method of embodiment 20 further comprising: receiving, at the one or more processors, an earnings rate goal for said team member for a current work period, said earnings rate goal being set by the team member, a supervisor, or generated automatically based on historical earnings rate data for said team member; calculating, on an ongoing basis, a progress metric reflecting a ratio of the most recently determined evaluated earnings rate to the earnings rate goal; and displaying the progress metric at the electronic display simultaneously with the graphic representing the evaluated earnings rate.

Embodiment 41—The method of embodiment 28 further comprising: receiving, during an active production activity and prior to completion of a pay period, an early access request from said team member requesting early access to a portion of the total earned pay accumulated to date during the active production activity; verifying that the requested early access amount does not exceed the total earned pay accumulated to date as reflected in the database; causing execution of an electronic transfer of the requested early access amount to a financial account designated by said team member; and updating the database to reflect the early access disbursement.

Embodiment 44—The method of embodiment 28 further comprising: receiving, from said team member, an automatic savings rule specifying that a predetermined percentage or amount of each payout is to be automatically transferred to a savings account designated by said team member; upon each payout execution, calculating the savings allocation based on the automatic savings rule; causing execution of an electronic transfer of the savings allocation to said savings account; and displaying a cumulative savings balance at the electronic display or a device of said team member.

Embodiment 45—The method of embodiment 28 further comprising: the plurality of payment options further comprises at least one benefit option enabling said team member to apply at least a portion of the earned pay for a pay period toward a benefit, said benefit comprising at least one of health insurance premium payment, retirement account contribution, transportation benefit, childcare benefit, or education benefit; and causing execution of the electronic transaction comprises electronically transferring the applied portion to a benefits administrator system associated with said benefit.

Embodiment 45—The method of embodiment 31 further comprising: generating, on an ongoing basis, a team evaluated earnings rate by aggregating the evaluated earnings rates of said team member and said additional team members forming a defined team; calculating a team earnings pool accumulating total earnings generated by said team during a current production period; and displaying the team evaluated earnings rate and the team earnings pool at the electronic display or another electronic display accessible to each of said team member and said additional team members.

Embodiment 46—The method of embodiment 45 further comprising: generating, on an ongoing basis, an opposing team evaluated earnings rate by aggregating evaluated earnings rates of a second defined team performing the production activity with additional machines; generating a team challenge display comparing the team earnings pool with an opposing team earnings pool; and displaying the team challenge display at one or more of the electronic displays accessible to members of both said team and said second defined team.

Embodiment 47—The method of embodiment 20 further comprising: maintaining, in the database, a set of one or more undisclosed earnings rate threshold values not displayed to said team member prior to being crossed; monitoring the most recently determined evaluated earnings rate against each of said undisclosed threshold values on an ongoing basis; and in response to the most recently determined evaluated earnings rate crossing one of said undisclosed threshold values for the first time, displaying a surprise achievement notification at the electronic display comprising a newly unlocked graphic and a designation of the crossed threshold value.

Embodiment 48—The method of embodiment 20 further comprising: in response to the most recently determined evaluated earnings rate crossing a threshold evaluated earnings rate such that a higher-tier graphic is selected, displaying an animated transition at the electronic display from the previously selected graphic to the newly selected graphic; and wherein the animated transition comprises a visual and/or auditory celebratory indicator displayed for a predetermined duration before settling on the newly selected graphic.

Embodiment 49—The method of embodiment 20 further comprising: determining, from historical earnings rate data for said team member stored in the database, a personalized earnings rate sensitivity profile comprising one or more threshold spacing values calibrated to the team member's historical earnings rate distribution; and setting the threshold evaluated earnings rates for said team member based on the personalized earnings rate sensitivity profile such that the spacing between thresholds reflects the team member's typical earnings rate variance, thereby maintaining challenge-skill balance in the visual feedback display.

Embodiment 50—The method of embodiment 20 further comprising: receiving, at the one or more processors, biometric data for said team member from a biometric monitoring device worn by said team member, said biometric data comprising at least one of heart rate, heart rate variability, galvanic skin response, or movement data; analyzing the biometric data to determine an engagement state of said team member, said engagement state comprising at least one of an active engagement state, a fatigue state, or a disengagement state; and in response to the determined engagement state, modifying at least one of the display of the graphic, the threshold evaluated earnings rates, or the display of coaching recommendations to maintain or restore the active engagement state.

Embodiment 51—The method of embodiment 20 further comprising: maintaining, in the database, historical production data for said team member indexed by time of day; analyzing the historical production data to determine one or more peak performance time windows for said team member during which the team member's evaluated earnings rate has historically been highest; displaying a peak performance window indicator at the electronic display prior to and during each peak performance time window; and adjusting the visual prominence or animation intensity of the graphic during peak performance time windows to maximize motivational salience during the team member's highest-productivity periods.

Embodiment 52—The method of embodiment 20 further comprising: generating, from historical earnings rate data stored in the database for said team member, an earnings identity profile comprising a personalized earnings tier designation reflecting the team member's historical earnings rate performance relative to a peer group; displaying the earnings identity profile designation at the electronic display during performance of the production activity as a persistent identity indicator alongside the evaluated earnings rate graphic; and updating the earnings identity profile designation in response to the team member's evaluated earnings rate performance over a predetermined period.

Embodiment 53—The method of embodiment 52 further comprising: maintaining, in the database, a cumulative lifetime earnings record for said team member comprising the total evaluated earnings generated across all pay periods during employment; calculating, on an ongoing basis, a projected lifetime earnings total based on the most recently determined evaluated earnings rate extrapolated across a predetermined career horizon; and displaying at the electronic display, simultaneously with the graphic representing the evaluated earnings rate, a visual representation of the cumulative lifetime earnings record and projected lifetime earnings total.

Embodiment 54—The method of embodiment 20 further comprising: maintaining, in the database, one or more community impact metrics linked to the team member's production output, said community impact metrics comprising at least one of a charitable contribution amount calculated as a percentage of evaluated earnings, a community fund contribution, or a social impact indicator; calculating the community impact metric on an ongoing basis based on the most recently determined evaluated earnings rate; and displaying the community impact metric at the electronic display simultaneously with the graphic representing the evaluated earnings rate.

Embodiment 55—The method of embodiment 20 further comprising: maintaining, in the database, a mastery progression model for said team member comprising a plurality of mastery levels each associated with a minimum sustained earnings rate threshold required to attain the level and a designation or reward associated therewith; determining a current mastery level for said team member based on the team member's evaluated earnings rate history; calculating a progress metric reflecting the team member's proximity to the next mastery level; and displaying the current mastery level, the next mastery level designation, and the progress metric at the electronic display simultaneously with the graphic representing the evaluated earnings rate.

Embodiment 56—The method of embodiment 31 further comprising: generating, on an ongoing basis, an anonymized or identified peer earnings rate distribution comprising the evaluated earnings rates of said additional team members ranked from highest to lowest; calculating a percentile rank for said team member within said peer earnings rate distribution; and displaying the percentile rank and a position indicator within the peer distribution at the electronic display simultaneously with the graphic representing the evaluated earnings rate.

Embodiment 57—The method of embodiment 31 further comprising: designating, in the database, a pairing relationship between said team member as an apprentice and one of said additional team members as a mentor based on the mentor's historically superior evaluated earnings rate performance; displaying, at the electronic display of said team member, the evaluated earnings rate of said mentor simultaneously with the team member's own evaluated earnings rate as a real-time pacing benchmark; and generating a progress indicator reflecting the gap between the team member's evaluated earnings rate and the mentor's evaluated earnings rate, updated on an ongoing basis during the production activity.

Embodiment 58—The method of embodiment 31 further comprising: generating, on an ongoing basis, a floor-wide evaluated earnings rate indicator reflecting the aggregate or average evaluated earnings rate across all team members actively performing the production activity; displaying the floor-wide evaluated earnings rate indicator at a shared electronic display accessible to all of said team members simultaneously; and updating the floor-wide indicator on an ongoing basis such that increases or decreases in collective productivity are immediately visible to all team members.

Embodiment 59—The method of embodiment 31 further comprising: receiving, at the one or more processors, evaluated earnings rates from a plurality of facilities or locations, each performing the same or comparable production activity; generating a cross-facility earnings rate ranking comparing the aggregate or top-performer evaluated earnings rate at each facility; and displaying the cross-facility ranking at the electronic display or a shared facility display on an ongoing basis during the production activity.

Embodiment 60—The method of embodiment 5 further comprising: in response to said team member exceeding the personal best evaluated earnings rate, generating a personal record notification; broadcasting the personal record notification to a shared electronic display accessible to other team members in the facility; and displaying on the shared electronic display the team member's name or identifier, the new personal best evaluated earnings rate, and a celebratory graphic for a predetermined display duration.

Embodiment 61—The method of embodiment 20 further comprising: determining a peripheral vision region of said team member's visual field based on the team member's orientation to the electronic display during performance of the production activity; displaying the graphic representing the evaluated earnings rate within the peripheral vision region of the electronic display such that the graphic is perceivable without requiring direct gaze at the electronic display; and selecting the graphic from the plurality of graphics based on the threshold evaluated earnings rate such that the selected graphic is distinguishable in peripheral vision by color, shape, or motion cues alone.

Embodiment 62—The method of embodiment 20 further comprising: limiting the information displayed at the electronic display during active performance of the production activity to: the graphic representing the evaluated earnings rate, a numerical display of the evaluated earnings rate, and not more than one additional contextual indicator selected from the group consisting of a streak count, a time remaining indicator, and a goal progress indicator; and suppressing display of non-essential information including historical data, settings interfaces, and administrative notifications during active performance of the production activity.

Embodiment 63—The method of embodiment 20 further comprising: in response to the most recently determined evaluated earnings rate crossing a threshold evaluated earnings rate, generating a non-visual sensory notification comprising at least one of: a haptic notification delivered to a wearable device of said team member, an auditory notification delivered through audio output associated with the electronic display or the wearable device, or a color change in ambient lighting associated with the team member's workstation; and configuring the non-visual sensory notification to be perceptible to said team member during performance of the production activity without requiring interruption of the production activity.

Embodiment 64—The method of embodiment 20 further comprising: calculating, on an ongoing basis, a current production pace metric reflecting the team member's rate of unit production over a most recent time window; calculating a target production pace required to maintain or achieve a target evaluated earnings rate; generating a pace guidance indicator reflecting whether the current production pace is above, at, or below the target production pace; and displaying the pace guidance indicator at the electronic display as a simple directional cue requiring minimal conscious attention to perceive and interpret.

Embodiment 65—The method of embodiment 7 further comprising: receiving, at the one or more processors, one or more retirement planning parameters for said team member comprising at least a target retirement age and a target retirement income; calculating a minimum evaluated earnings rate required to achieve the retirement target given the team member's current age, current savings, and projected earnings trajectory; displaying the minimum retirement-required earnings rate as a threshold indicator at the electronic display simultaneously with the graphic representing the evaluated earnings rate; and updating the retirement readiness indicator on an ongoing basis to reflect changes in the evaluated earnings rate.

Embodiment 66—The method of embodiment 28 further comprising: calculating, on an ongoing basis, a net evaluated earnings rate for said team member by applying one or more tax rate parameters or deduction parameters to the evaluated earnings rate to generate a net-of-tax evaluated earnings rate; and displaying the net evaluated earnings rate simultaneously with the gross evaluated earnings rate at the electronic display, with the graphic selected based on the gross evaluated earnings rate.

Embodiment 67—The method of embodiment 20 further comprising: calculating, on an ongoing basis, an earnings rate volatility metric reflecting the standard deviation or range of the team member's evaluated earnings rate over a predetermined rolling time window; displaying the earnings rate volatility metric at the electronic display as a stability indicator reflecting the consistency of the team member's production pace; and generating a consistency achievement notification in response to the earnings rate volatility metric falling below a predetermined stability threshold, indicating sustained consistent performance.

Embodiment 68—The method of embodiment 7 further comprising: receiving, at the one or more processors, one or more family financial goal parameters for said team member comprising at least a goal description, a target savings amount, and a target achievement date; calculating, on an ongoing basis, a family goal progress metric reflecting the portion of the target savings amount funded by the team member's accumulated earnings to date and the projected additional time to achieve the goal at the current evaluated earnings rate; displaying the family goal progress metric at the electronic display simultaneously with the graphic representing the evaluated earnings rate, such that the team member can perceive the real-time connection between current production pace and family goal achievement.

Embodiment 69—The method of embodiment 28 further comprising: receiving, from said team member, an education savings rule specifying that a predetermined percentage or fixed amount of each payout is to be automatically transferred to a designated education savings account, including a tax-advantaged education savings account, for a designated beneficiary; upon each payout execution, calculating the education savings allocation based on the education savings rule; causing execution of an electronic transfer of the education savings allocation to said education savings account; and displaying a cumulative education savings balance and a projected education fund value at a target date at the electronic display or a device of said team member.

Embodiment 70—The method of embodiment 7 further comprising: receiving, at the one or more processors, home purchase goal parameters comprising at least a target home purchase price, a required down payment percentage, and a target purchase timeline; calculating, on an ongoing basis, a down payment progress metric reflecting the accumulated savings attributable to the team member's earnings and the additional production required at the current evaluated earnings rate to achieve the required down payment by the target timeline; displaying the down payment progress metric at the electronic display simultaneously with the graphic representing the evaluated earnings rate.

Embodiment 71—The method of embodiment 20 further comprising: generating, at the one or more processors, a family portal access credential for one or more designated family members of said team member; transmitting, via a network, real-time earnings data comprising the evaluated earnings rate and cumulative earnings for the current pay period to a family portal application accessible via the family portal access credential on an electronic device of said designated family members; displaying, at the family portal application, a real-time visualization of the evaluated earnings rate and cumulative earnings; and receiving, at the one or more processors, a family encouragement message from said designated family member and transmitting the encouragement message to the electronic display of said team member during performance of the production activity.

Embodiment 72—The method of embodiment 20 further comprising: retrieving, from a regulatory database, a minimum wage rate applicable to said team member based on the team member's jurisdiction, employment classification, and work period; calculating, on an ongoing basis, a minimum wage compliance metric reflecting whether the evaluated earnings rate meets or exceeds the applicable minimum wage rate; in response to the minimum wage compliance metric indicating that the evaluated earnings rate has fallen below the applicable minimum wage rate, generating a compliance alert displayed at the electronic display and transmitted to a supervisor electronic device; and automatically adjusting the unit of output based earnings rate for said team member to ensure the evaluated earnings rate meets the minimum wage threshold when the production pace would otherwise result in a sub-minimum wage outcome.

Embodiment 73—The method of embodiment 28 further comprising: retrieving, from a regulatory or employer database, overtime threshold parameters applicable to said team member comprising at least a weekly hours threshold and an overtime earnings multiplier; calculating, on an ongoing basis, a projected hours accumulation for the current work period based on elapsed and projected work time; in response to the projected hours accumulation approaching the overtime threshold within a predetermined margin, displaying an overtime approaching notification at the electronic display comprising the current earnings rate, the projected overtime earnings rate, and the additional compensation available upon crossing the overtime threshold; and recalculating and displaying the evaluated earnings rate using the overtime multiplier upon crossing the overtime threshold.

Embodiment 74—The method of embodiment 72 further comprising: maintaining, in the database, a multi-jurisdiction regulatory table comprising minimum wage rates, overtime rules, break requirements, and maximum hours limits for a plurality of jurisdictions; determining the applicable jurisdiction for said team member based on the team member's work location, employment agreement, or other applicable parameters; dynamically applying the applicable jurisdiction's regulatory parameters to the minimum wage compliance calculation and the overtime threshold calculation on an ongoing basis; and generating a compliance report reflecting the team member's evaluated earnings rate history relative to applicable regulatory requirements for each work period, said compliance report accessible to the employer and available for regulatory audit purposes.

Embodiment 75—The method of embodiment 55 further comprising: maintaining, in the database, a skills credentialing framework comprising a plurality of credential levels each associated with a minimum sustained evaluated earnings rate threshold maintained over a predetermined qualification period; monitoring the team member's evaluated earnings rate history to determine whether the team member has sustained the evaluated earnings rate at or above the minimum threshold for the required qualification period; in response to the team member satisfying the credential requirements, automatically generating and issuing a digital skills credential to the team member comprising the credential level, the qualifying earnings rate, and the qualification period; and transmitting the digital skills credential to the team member's electronic device and recording the credential in the database.

Embodiment 76—The method of embodiment 75 further comprising: maintaining, in the database, an apprenticeship progression framework comprising a plurality of apprenticeship levels each associated with an earnings rate threshold required for advancement and an earnings rate-linked compensation rate applicable at each level; automatically advancing said team member to the next apprenticeship level upon satisfaction of the earnings rate threshold for the required qualification period; updating the unit of output based earnings rate for said team member upon advancement to reflect the compensation rate applicable at the new apprenticeship level; and displaying an apprenticeship advancement notification at the electronic display and updating the graphic selection thresholds to reflect the new earnings rate tier structure applicable at the advanced level.

Embodiment 77—The method of embodiment 75 further comprising: generating a portable digital earnings credential comprising the team member's verified earnings rate performance history, credential levels achieved, and qualifying production data, said credential encoded in a standardized digital format accessible to prospective employers; transmitting the portable digital earnings credential, upon authorization by said team member, to a credential verification system accessible to one or more prospective employers; and receiving, at the one or more processors, a credential verification request from a prospective employer system and responding with verification of the team member's earnings rate credential.

Embodiment 78—The method of embodiment 16 further comprising: detecting, using the artificial intelligence model, an earnings rate plateau condition for said team member comprising a sustained period during which the evaluated earnings rate has not materially increased despite the team member's continued effort; analyzing the earnings rate plateau condition to identify one or more skill gaps or production inefficiencies contributing to the plateau; generating a training recommendation comprising one or more specific training modules, techniques, or resources directed to addressing the identified skill gaps; and displaying the training recommendation at the electronic display of the team member and transmitting a training referral to a supervisor or training management system.

Embodiment 79—The method of embodiment 20 further comprising: receiving, at the one or more processors, an accessibility profile for said team member comprising one or more accessibility parameters selected from the group consisting of: visual impairment parameters, color vision deficiency parameters, cognitive processing parameters, and motor impairment parameters; selecting, based on the accessibility profile, an adapted display configuration comprising at least one of: a high-contrast graphic set optimized for the team member's visual parameters, an audio narration of the evaluated earnings rate and graphic tier, a simplified graphic set with reduced visual complexity, or an enlarged display format; and displaying and updating the graphic representing the evaluated earnings rate using the adapted display configuration.

Embodiment 80—The method of embodiment 49 further comprising: receiving, at the one or more processors, a neurodiversity profile for said team member indicating attention-deficit characteristics; generating, based on the neurodiversity profile, an ADHD-optimized display configuration comprising: increased graphic animation frequency, shorter threshold intervals to increase the rate of visual feedback events, a task-segmentation display dividing the production period into shorter gamified sub-periods each with their own graphic tier progression, and heightened visual and haptic notifications at each sub-period completion; and displaying and updating the earnings rate graphic using the ADHD-optimized display configuration.

Embodiment 81—The method of embodiment 20 further comprising: maintaining, in the database, a pre-injury or pre-leave baseline evaluated earnings rate for said team member established prior to a medical leave, injury, or extended absence; calculating, on an ongoing basis during the team member's return-to-work period, a recovery progress metric reflecting the team member's current evaluated earnings rate as a percentage of the pre-leave baseline; displaying the recovery progress metric at the electronic display simultaneously with the graphic representing the evaluated earnings rate, providing the team member with real-time feedback on their return-to-work progress; and generating milestone notifications at the electronic display in response to the team member's evaluated earnings rate recovering to predetermined percentages of the baseline.

Embodiment 82—The method of embodiment 39 further comprising: receiving, at the one or more processors, one or more environmental performance parameters for the production activity comprising at least one of: a material waste rate, an energy consumption rate, a recycled content percentage, or a carbon emission rate per unit of output; calculating a green production bonus earnings rate adjustment based on the environmental performance parameters meeting or exceeding one or more environmental performance thresholds; adding the green production bonus earnings rate adjustment to the evaluated earnings rate to generate a green-adjusted evaluated earnings rate; and displaying the green-adjusted evaluated earnings rate at the electronic display simultaneously with the environmental performance parameters, such that the team member can perceive the real-time connection between environmental performance and earnings rate.

Embodiment 83—The method of embodiment 28 further comprising: calculating, based on the production activity's environmental performance parameters, a carbon offset contribution amount as a function of the total earned pay for the pay period and a carbon offset contribution rate selected by said team member; upon payout execution, causing execution of an electronic transfer of the carbon offset contribution amount to a verified carbon offset fund or registry; and displaying a cumulative carbon offset contribution at the electronic display or a device of said team member, said cumulative contribution expressed in both monetary terms and equivalent environmental impact units.

Embodiment 84—The method of embodiment 47 further comprising: maintaining, in the database, one or more sustainability milestone thresholds reflecting cumulative environmental performance achievements for said team member, comprising at least one of: a cumulative waste reduction target, a cumulative energy savings target, or a cumulative carbon offset target; monitoring the team member's cumulative environmental performance against each sustainability milestone threshold; and in response to the team member's cumulative environmental performance crossing a sustainability milestone threshold, displaying an environmental achievement notification at the electronic display and unlocking a sustainability-themed graphic from the plurality of graphics for inclusion in the earnings rate display

Embodiment 85—The method of embodiment 32 further comprising: aggregating evaluated earnings rate data from a plurality of franchise locations performing the same or comparable production activity; generating a franchise-wide earnings rate benchmark comprising at least a median evaluated earnings rate, a top-quartile evaluated earnings rate, and a bottom-quartile evaluated earnings rate across said plurality of franchise locations; transmitting the franchise-wide earnings rate benchmark to each franchise location's supervisor electronic display; and displaying, at each franchise location's supervisor electronic display, the location's aggregate evaluated earnings rate relative to the franchise-wide benchmark on an ongoing basis.

Embodiment 86—The method of embodiment 85 further comprising: generating, at the one or more processors, a franchisor-level aggregate analytics dashboard comprising: aggregate evaluated earnings rates across all franchise locations, workforce productivity trends over time, correlation analyses between earnings rate and business performance metrics, and identification of top-performing and underperforming franchise locations based on evaluated earnings rate data; transmitting the franchisor-level dashboard to a franchisor electronic system; and updating the franchisor-level dashboard on a predetermined periodic basis.

Embodiment 87—The method of embodiment 85 further comprising: upon deployment of the system at a new franchise location, initiating a calibration period during which production data and evaluated earnings rates are collected for team members at said new franchise location; generating, upon completion of the calibration period, a location-specific earnings rate baseline and threshold configuration calibrated to the production characteristics of said new franchise location; and automatically configuring the graphic selection threshold evaluated earnings rates for said new franchise location based on the location-specific baseline.

Embodiment 88—A computerized system for providing real-time earnings rate visualization as a licensed platform service, the system comprising: one or more processors configured to receive, via an application programming interface, production data from a third-party production management system, earnings rate parameters from a third-party compensation system, and display configuration parameters from a third-party employer; a visualization engine configured to generate, on an ongoing basis, an evaluated earnings rate for one or more workers by multiplying the received production data with the received earnings rate parameters; a graphic selection engine configured to select, from a plurality of graphics each associated with a threshold evaluated earnings rate, the graphic associated with the highest surpassed threshold based on the most recently determined evaluated earnings rate; and a transmission engine configured to transmit the selected graphic and the evaluated earnings rate to one or more electronic display devices designated by the third-party employer.

Embodiment 89—The method of embodiment 16 further comprising: analyzing, using the artificial intelligence model, the team member's evaluated earnings rate history over a predetermined rolling period to detect one or more burnout risk indicators comprising at least one of: a sustained decline in earnings rate from a prior peak, an increase in earnings rate variability relative to historical norms, or a pattern of earnings rate decline in the latter portion of successive work periods; generating a burnout risk score for said team member based on the detected burnout risk indicators; and in response to the burnout risk score exceeding a predetermined threshold, generating a wellbeing alert transmitted to a supervisor electronic device and displaying a wellness check-in prompt at the electronic display of said team member.

Embodiment 90—The method of embodiment 89 further comprising: in response to the burnout risk score exceeding a critical threshold or upon detection of a regulatory-required break period, automatically pausing the evaluation and display of the evaluated earnings rate; displaying, at the electronic display, a mandatory recovery notification comprising the duration of the required pause and a wellness message; resuming the evaluation and display of the evaluated earnings rate upon expiration of the pause period; and recording the pause event in the database for compliance and wellbeing monitoring purposes.

Embodiment 91—The method of embodiment 5 further comprising: maintaining, in the database, a library of positive affirmations associated with earnings rate performance levels, said affirmations personalized to said team member based on historical earnings rate performance and demographic profile; generating, on a predetermined periodic basis during the production activity, a contextually appropriate affirmation selected from the library based on the most recently determined evaluated earnings rate; displaying the affirmation at the electronic display for a predetermined duration alongside the graphic representing the evaluated earnings rate; and updating the affirmation library based on the team member's response patterns to prior affirmations.

Embodiment 92—The method of embodiment 64 further comprising: maintaining, in the database, a fatigue model for said team member derived from historical production data reflecting the team member's typical earnings rate decline pattern across a work period; calculating, on an ongoing basis, a recommended production pace for each segment of the current work period based on the fatigue model and the remaining time in the work period, said recommended production pace designed to maximize total earnings for the complete work period while accounting for fatigue-related production decline; and displaying the recommended production pace as a pacing recommendation at the electronic display, updated throughout the work period.

Embodiment 93—The method of embodiment 20 further comprising: receiving, at the one or more processors, employer profitability parameters comprising at least a per-unit revenue amount and a per-unit cost structure for the production activity; calculating, on an ongoing basis, a per-unit employer margin contribution generated by the team member's production at the current evaluated earnings rate; calculating a profit share amount for said team member as a function of the per-unit employer margin contribution and a profit share percentage; adding the profit share amount to the evaluated earnings rate to generate a total compensation rate; and displaying the total compensation rate, the base evaluated earnings rate, and the profit share component simultaneously at the electronic display.

Embodiment 94—The method of embodiment 31 further comprising: generating, at the one or more processors, an internal task market display comprising a plurality of available production tasks each associated with a unit of output based earnings rate, a difficulty rating, and an estimated earnings rate range based on historical performance data for said task; receiving, from said team member, a bid or selection for one of said available production tasks; assigning the selected production task to said team member upon acceptance of the bid or selection; and initiating the evaluated earnings rate calculation for said team member using the earnings rate associated with the selected task.

Embodiment 95—The method of embodiment 93 further comprising: calculating, on an ongoing basis, a labor cost savings amount representing the reduction in per-unit labor cost achieved by the team member's current evaluated earnings rate relative to a baseline labor cost rate; calculating a cumulative labor cost savings amount for the current pay period; and displaying the cumulative labor cost savings amount at the electronic display simultaneously with the evaluated earnings rate, framed as the team member's contribution to employer cost savings.

Embodiment 96—The method of embodiment 31 further comprising: receiving, at the one or more processors, a challenge initiation from said team member directed to one of said additional team members, said challenge comprising a specified challenge period and a specified earnings rate target; transmitting the challenge to the challenged team member's electronic display and receiving an acceptance or declination; upon acceptance, monitoring the evaluated earnings rates of both the challenging and challenged team members against the specified earnings rate target for the specified challenge period; and displaying a real-time challenge scoreboard at the electronic displays of both team members comprising both evaluated earnings rates, a progress indicator, and a remaining time display.

Embodiment 97—The method of embodiment 20 further comprising: retrieving, at the one or more processors, a current currency exchange rate for a target currency designated by said team member from a currency data source; converting the evaluated earnings rate from a base currency to the target currency using the retrieved exchange rate on an ongoing basis; calculating a purchasing power equivalent for the converted evaluated earnings rate based on a cost-of-living index for a geographic location designated by said team member; and displaying the evaluated earnings rate in both the base currency and the target currency, and the purchasing power equivalent, simultaneously at the electronic display.

Embodiment 98—The method of embodiment 75 further comprising: receiving, at the one or more processors, verified military service performance data for said team member comprising at least one of: military occupational specialty performance ratings, physical fitness assessment scores, leadership evaluation metrics, or technical proficiency ratings; mapping the military service performance data to an initial earnings rate tier in the earnings rate framework based on a military-to-civilian performance equivalency table stored in the database; initializing the team member's earnings rate threshold configuration and mastery progression starting point based on the mapped initial tier; and displaying a military-to-civilian performance bridge notification at the electronic display acknowledging the team member's military service performance as the foundation for their civilian earnings rate starting point.

Embodiment 99—The method of embodiment 20 further comprising: receiving, at the one or more processors, a workforce reintegration designation for said team member indicating participation in a workforce reintegration program for individuals returning to employment following incarceration, long-term unemployment, or other extended workforce absence; initiating a reintegration earnings rate protocol comprising: a reduced initial threshold configuration providing more frequent positive feedback events during the reintegration period, a progressive threshold expansion schedule increasing challenge over a predetermined reintegration timeline, and a reintegration milestone display tracking the team member's progress through the reintegration protocol; and displaying the reintegration milestone progress simultaneously with the evaluated earnings rate graphic at the electronic display.

Embodiment 100—A computerized method for increasing engagement in a production activity, said method comprising: generating production data at least in part by comparing captured image data of units of output against build files for units of output produced by binder jetting machines (“machines”), the captured image data being captured by cameras, said cameras forming part of data acquiring devices, said cameras and data acquiring devices forming part of augmented reality devices, during performance of a production activity at the machines for generating the units of output using the build files; acquiring the production data from the machines on an ongoing basis while the production activity is underway by way of the data acquiring devices; retrieving unit of output based earnings rate data for team members tasked with performing the production activity with the machines from a database; generating, on an ongoing basis while performance of the production activity is underway, an evaluated earnings rate for each of said team members by multiplying the production data with the output based earnings rate for said team members on a machine specific basis; displaying and updating, on an ongoing basis while the production activity is underway, graphics at an electronic displays representing the evaluated earnings rate; and selecting the graphics from a plurality of graphics, wherein the graphics are automatically selected on a team member specific basis from a plurality of graphics, each of said plurality of graphics being associated with a threshold evaluated earnings rate, the selected graphics being the graphics of the plurality of candidate graphics associated with a highest surpassed one of the threshold evaluated earning rates based on a most recently determined evaluated earnings rate on the team member specific basis.

Embodiment 101—A computerized method for increasing engagement in a production activity, said method comprising: acquiring production data for a production activity from machines for performing the production activity to generate units of output, said production data acquired on an ongoing basis while the production activity is underway by way of data acquiring devices forming part of, or in electronic association with, the machines; retrieving unit of output based earnings rate data for team members tasked with performing the production activity with the machines from a database; generating, on an ongoing basis while performance of the production activity is underway, an evaluated earnings rate for each of said team members by multiplying the production data with the output based earnings rate for said team members on a machine specific basis; displaying and updating, on an ongoing basis while the production activity is underway, graphics at an electronic displays representing the evaluated earnings rate; selecting the graphics from a plurality of graphics, wherein the graphics are automatically selected on a team member specific basis from a plurality of graphics, each of said plurality of graphics being associated with a threshold evaluated earnings rate, the selected graphics being the graphics of the plurality of candidate graphics associated with a highest surpassed one of the threshold evaluated earning rates based on a most recently determined evaluated earnings rate on the team member specific basis; generating a ranking of said team member and each of additional team members based on the evaluated earnings rate for the team member and the evaluated earnings rates for the additional team members; and causing the ranking to be displayed at the electronic display or another electronic display.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

Certain operations described herein may be performed by one or more electronic devices. Each electronic device may comprise one or more processors, electronic storage devices, executable software instructions, and the like configured to perform the operations described herein. The electronic devices may be general purpose computers of specialized computing device. The electronic devices may be personal computers, smartphone, tablets, databases, servers, or the like. The electronic connections described herein may be accomplished by wired or wireless means.

What is claimed is:

1. A computerized method for providing real-time earnings feedback to a worker performing a production or service activity, the method implemented by one or more processors, said method comprising:
acquiring performance data for the production or service activity performed by the worker, said performance data comprising a count of output units completed by the worker, said performance data acquired on an ongoing basis while the production or service activity is underway by way of a data acquiring device associated with the production or service activity;
retrieving a unit of output based earnings rate for the worker from a database;
generating, on an ongoing basis while the production or service activity is underway, an evaluated earnings rate for said worker by multiplying the performance data with the unit of output based earnings rate; and
displaying and updating, on an ongoing basis, a graphic at an electronic display associated with the worker, said graphic representing the evaluated earnings rate and automatically selected from a plurality of graphics each associated with a threshold evaluated earnings rate.

2. The method of claim 1 wherein:
the electronic display is part of a wearable electronic device.

3. The method of claim 2 wherein:
the wearable electronic device comprises a watch-style device adapted to secure around a wrist of said worker.

4. The method of claim 2 wherein:
the wearable electronic device comprises an augmented reality headset worn by said worker; and
the graphic representing the evaluated earnings rate is displayed as an overlay on a view of the worker's workspace rendered by the augmented reality headset, said overlay positioned within the worker's field of view during performance of the production or service activity.

5. The method of claim 1 wherein:
the production or service activity is a production activity; and
the electronic display is provided at a machine for performing the production activity to generate the output units.

6. The method of claim 5 wherein:
the production activity is a manufacturing activity;
the output units are tangible objects; and
the machine is an item of industrial equipment for producing the tangible objects.

7. The method of claim 6 wherein:
the industrial equipment is an additive manufacturing machine.

8. The method of claim 1 further comprising:
simultaneously transmitting the evaluated earnings rate to a plurality of electronic devices associated with said worker, said plurality of electronic devices comprising at least two of: the electronic display, a wearable electronic device secured to the body of the worker, and a mobile device of the worker; and
displaying and updating the graphic representing the evaluated earnings rate substantially simultaneously across each of said plurality of electronic devices.

9. The method of embodiment 1 further comprising:
maintaining, in the database, a record of a personal best evaluated earnings rate for said worker over a predetermined historical period;
comparing the most recently determined evaluated earnings rate against the personal best evaluated earnings rate; and
in response to the most recently determined evaluated earnings rate exceeding the personal best evaluated earnings rate, displaying a milestone achievement notification at the electronic display, and updating the personal best evaluated earnings rate stored in the database.

10. The method of claim 1 further comprising:
tracking a consecutive-period streak count reflecting a number of successive pay periods during which said worker's evaluated earnings rate exceeded a predetermined earnings rate target;
displaying the consecutive-period streak count at the electronic display during performance of the production activity; and
in response to the consecutive-period streak count reaching a streak threshold, displaying a streak achievement notification at the electronic display.

11. The method of embodiment 1 further comprising:
calculating, on an ongoing basis while the production activity is underway, a projected total earnings amount for a current pay period based on the most recently determined evaluated earnings rate and a remaining time in the current pay period; and
displaying the projected total earnings amount at the electronic display with the graphic representing the evaluated earnings rate.

12. The method of embodiment 11 further comprising:
receiving, at the one or more processors, one or more investment parameters including at least an investment return rate and an investment duration;
calculating a projected investment value of the projected total earnings amount after the investment duration at the investment return rate; and
displaying the projected investment value at the electronic display with the projected total earnings amount.

13. The method of claim 1 wherein:
the production or service activity is a service activity; and
the output units are service units.

14. The method of claim 13 wherein:
the service activity comprises a healthcare service activity;
the service units comprise at least one of patient visits completed, procedures performed, prescriptions dispensed, or assessments documented; and
the data acquiring device comprises an electronic health record system, patient scheduling system, or automated dispensing system in electronic communication with the one or more processors.

15. The method of embodiment 13 wherein:
the service activity comprises a logistics or delivery activity;
the service units comprise at least one of packages scanned, deliveries completed, routes finished, or pick-and-place operations performed; and
the data acquiring device comprises at least one of a barcode scanner, RFID reader, GPS tracking device, or warehouse management system in electronic communication with the one or more processors.

16. The method of claim 13 wherein:
the service activity comprises a food service or restaurant activity;
the service units comprise at least one of orders fulfilled, meals prepared, tables served, or transactions completed; and
the data acquiring device comprises a point-of-sale system, kitchen display system, or order management system in electronic communication with the one or more processors.

17. The method of claim 1 wherein:
the production or service activity comprises on-demand work activities;
the service units comprise on-demand work activities completed; and
the data acquiring device comprises gig platform for connecting the workers with hiring parties for the on-demand work activities.

18. A computerized method for coaching a team member to increase an evaluated earnings rate during a production activity, the method implemented by one or more processors, said method comprising:
acquiring production data for a production activity from a machine on an ongoing basis while the production activity is underway;
generating, on an ongoing basis, an evaluated earnings rate for a team member performing the production activity by multiplying the production data with a unit of output based earnings rate for the team member;
analyzing the evaluated earnings rate using an artificial intelligence model trained on historical production data and historical evaluated earnings rates for the team member or a plurality of team members to identify production patterns associated with higher evaluated earnings rates;
generating, based on said analysis, one or more coaching recommendations for the team member directed to increasing the evaluated earnings rate; and
transmitting the one or more coaching recommendations to an electronic device of the team member.

19. The method of embodiment 18 further comprising:
displaying the one or more coaching recommendations at the electronic device simultaneously with the graphic representing the evaluated earnings rate such that the coaching recommendations are presented to the team member in the context of the current earnings rate; and
updating the one or more coaching recommendations on an ongoing basis during the production activity as the evaluated earnings rate changes.

20. The method of embodiment 18 further comprising:
analyzing, using the artificial intelligence model, historical production data for said team member to identify an earnings rate ceiling representing the team member's historical maximum sustained evaluated earnings rate;
generating a visual indicator of the earnings rate ceiling at the electronic display showing a threshold between the team member's current evaluated earnings rate and the earnings rate ceiling; and
in response to the most recently determined evaluated earnings rate approaching the earnings rate ceiling within a predetermined margin, displaying a breakthrough notification at the electronic display prompting the team member to sustain or increase output to surpass the earnings rate ceiling.

21. The method of embodiment 18 further comprising:
acquiring production data and generating evaluated earnings rates for a plurality of team members performing the production activity;
analyzing the evaluated earnings rates for said plurality of team members using the artificial intelligence model to identify team-level production patterns associated with higher aggregate evaluated earnings rates;
generating team-level coaching recommendations directed to increasing aggregate evaluated earnings rates across the plurality of team members; and
transmitting the team-level coaching recommendations to an electronic device of a supervisor or manager associated with the plurality of team members.
